(19) United States
(12) Patent Application Publication
(10) Pub. No.: US 2024/0115820 A1
(43) Pub. Date: Apr. 11, 2024

(54) SENSOR ASSEMBLY FOR RESPIRATORY APPARATUS

(71) Applicant: ResMed Pty Ltd, Bella Vista, NSW (AU)

(72) Inventors: Hayat CHAMTIE, Sydney (AU); Simon Robert CORK, Wollstonecraft (AU); Benjamin John LEAVENS, Woollahra (AU); Sudeep SINGH, Sydney (AU); Millen James RATH-MAY, Sydney (AU); Benjamin Mark Moffat DRAYTON, Sydney (AU)

(73) Assignee: ResMed Pty Ltd, Bella Vista, NSW (AU)

(21) Appl. No.: 17/768,771

(22) PCT Filed: Oct. 23, 2020
(86) PCT No.: PCT/AU2020/051145

§ 371 Date: Apr. 13, 2022

(51) Int. Cl. A61M 16/00; A61M 16/10

(52) U.S. Cl. CPC A61M 16/0066; A61M 16/0003; A61M 16/1055; A61M 16/107; A61M 16/109; A61M 2016/0024; A61M 2016/0027; A61M 2205/502

(57) ABSTRACT

A sensor module assembly for a respiratory therapy apparatus may be configured for a seal-based securement within a respiratory therapy device that includes a separable pneumatic block to make assembly or replacement more efficient. The sensor module assembly may include a flow manifold. The flow manifold may have an inlet and an outlet and a main pneumatic path between the inlet and the outlet. The assembly may have a sensor module with sensor(s) on a printed circuit board. The sensor module may be pneumatically and fixedly coupled to the flow manifold. The inlet of the flow manifold may be configured for removable coupling with an outlet of the pneumatic block assembly. Moreover, the outlet of the flow manifold may be configured for removable coupling with a pneumatic path of a housing panel of the respiratory therapy apparatus. The pneumatic path is adapted for supplying breathable gas to a patient interface.

### 1 CROSS-REFERENCE TO RELATED APPLICATIONS

This application claims the benefit of U.S. Provisional Application No. 62/926,028, filed 25 Oct. 2020, the entire disclosure of which is hereby incorporated herein by reference.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use, such as methods and devices useful for measuring gas characteristics of a flow of a breathable gas provided for a respiratory therapy such as a therapy generated by a respiratory pressure therapy apparatus such as a ventilator.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the air into the venous blood and carbon dioxide to move out. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2011.

A range of respiratory disorders exist. Certain disorders may be characterised by particular events, e.g. apneas, hypopneas, and hyperpneas.

Obstructive Sleep Apnea (OSA), a form of Sleep Disordered Breathing (SDB), is characterized by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory Failure is an umbrella term for respiratory disorders in which patients are unable to ventilate enough to balance the CO₂ in their blood if their metabolic activity rises much above rest. Respiratory failure encompasses all of the following conditions.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD, and Chest Wall disorders. In some forms, the comfort and effectiveness of these therapies may be improved.

Patients receiving non-invasive ventilation, particularly when asleep and/or under sedation, are often subject to upper airway instability and collapse, as in OSA. Such instability and collapse can compromise the effectiveness of the ventilation therapy by reducing or even nullifying the pressure actually reaching the lungs from the ventilator.

The upper airway can be stabilised by maintaining a positive base pressure, referred to herein as the EPAP, upon which ventilatory assistance is superimposed. An insufficient EPAP permits upper airway collapse, while an excessive EPAP may fully stabilise the upper airway but negatively impact on comfort, promote mask leak, or pose cardiovascular complications. The task of choosing an EPAP that is sufficient to generally maintain upper airway stability across the range of sleep states, posture, level of sedation, and progression of disease while avoiding negative side-effects (a task known as EPAP titration) is a significant challenge even for experienced clinicians with the benefit of a full polysomnographic (PSG) study. An appropriately titrated EPAP is a balance between extremes, not necessarily one that prevents all obstructive events. While NW enjoys growing usage globally, only a fraction of patients are administered NIV with the benefit of a PSG study to titrate the EPAP. In more acute environments, historically there is limited awareness of the effects of sleep and sedation on the efficacy of non-invasive ventilation.

There is therefore a significant need for NIV therapies capable of automatically adjusting the EPAP (i.e. performing “EPAP auto-titration”) in dynamic response to the changing condition of an NIV patient's upper airway.

### 2.2.3 Treatment Systems

These therapies may be provided by a treatment system or device. Such systems and devices may also be used to diagnose a condition without treating it.

A treatment system may comprise a Respiratory Therapy Device (RPT device), an air circuit, a humidifier, a patient interface, and data management.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O.

### 2.2.3.2 Respiratory Therapy (RPT) Device

Air pressure generators are known in a range of applications, e.g. industrial-scale ventilation systems. However, air pressure generators for medical applications have particular requirements not fulfilled by more generalised air pressure generators, such as the reliability, size and weight requirements of medical devices. In addition, even devices designed for medical treatment may suffer from shortcomings, pertaining to one or more of: comfort, noise, ease of use, efficacy, size, weight, manufacturability, cost, and reliability.

One known RPT device used for treating sleep disordered breathing is the S9 Sleep Therapy System, manufactured by ResMed Limited. Another example of an RPT device is a ventilator. Ventilators such as the ResMed Stellar™ Series of Adult and Paediatric Ventilators may provide support for invasive and non-invasive non-dependent ventilation for a range of patients for treating a number of conditions such as but not limited to NMD, OHS and COPD.

The ResMed Elisée™ 150 ventilator, ResMed Astral, and ResMed VS III™ ventilator may provide support for invasive and non-invasive dependent ventilation suitable for adult or paediatric patients for treating a number of conditions. These ventilators provide volumetric and barometric ventilation modes with a single or double limb circuit. RPT devices typically comprise a pressure generator, such as a motor-driven or motor-operated blower or a compressed gas reservoir, and are configured to supply a flow of air to the airway of a patient. In some cases, the flow of air may be supplied to the airway of the patient at positive pressure such as with a pressure control loop of a controller according to a pressure set point or a flow rate control loop of a controller according to a flow rate set point. The outlet of the RPT device is connected via an air circuit to a patient interface such as those described above. Such a device may typically include one or more sensors for measuring gas characteristics of the generated breathable gas that is provided for the particular therapy. The gas characteristics can be monitored and processed for determining information about the patient and/or may serve as part of one or more control loops of the therapy controller.

RPT devices may include for example, a high flow therapy device configured to provide a high flow therapy. In this regard, some respiratory therapies may aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO₂ from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

### 2.2.3.3 Humidifier

Delivery of a flow of air without humidification may cause drying of airways. The use of a humidifier with an RPT device and the patient interface produces humidified gas that minimizes drying of the nasal mucosa and increases patient airway comfort. In addition in cooler climates, warm air applied generally to the face area in and about the patient interface is more comfortable than cold air. A range of artificial humidification devices and systems are known, however they may not fulfil the specialised requirements of a medical humidifier.

It may be desirable to develop further assemblies for such RPT devices, such as a ventilator that may improve existing devices such as to improve the sensor placement or arrangement within a housing for such a device. For example, placement or arrangements may improve manufacture and/or maintenance such as by making it easier to install and/or replace such sensors.

### 3 BRIEF SUMMARY OF THE TECHNOLOGY

The present technology is directed towards providing medical devices that may be used in the screening, diagnosis, monitoring of and/or treating respiratory disorders having one or more of improved comfort, cost, efficacy, ease of use and manufacturability.

Some versions of the present technology may include a sensor assembly that is formed as a module.

Some versions of the present technology include a sensor module assembly for a respiratory therapy apparatus that includes a pneumatic block assembly. The sensor module assembly may include a flow manifold. The flow manifold may include an inlet and an outlet and a main pneumatic path between the inlet and the outlet. The sensor module assembly may include a sensor module. The sensor module may include a plurality of sensors on a printed circuit board. The sensor module may be pneumatically and fixedly coupled to the flow manifold.

In some versions, the sensor module assembly may be configured to be substantially secured for use within the respiratory therapy apparatus by its pneumatic couplings. In some versions, the inlet of the flow manifold may be configured for removable coupling with an outlet associated with the pneumatic block assembly. In some versions, the outlet of the flow manifold may be configured for removable coupling with a pneumatic path associated with a housing structure of the respiratory therapy apparatus. The pneumatic path may be adapted for supplying breathable gas to a patient interface.

In some versions, the inlet of the flow manifold may be configured for removable coupling with an outlet of the pneumatic block assembly. In some versions, the outlet of the flow manifold may be configured for removable coupling with a pneumatic path of a housing cover panel of the respiratory therapy apparatus. The pneumatic path may be adapted for supplying breathable gas to a patient interface.

In some versions, the flow manifold may include a cylinder. The cylinder may include the main pneumatic path. The cylinder may have a central axis from the inlet of the flow manifold to the outlet of the flow manifold. The central axis may be along a length of the cylinder. The flow manifold may further include a plurality of gas ports. The plurality of gas ports may be pneumatically coupled to the main pneumatic path. Each of the plurality of gas ports may be cylindrical. Each cylindrical gas port may have a central axis. The central axes of the cylindrical gas ports may be substantially parallel. The central axes of the cylindrical gas ports may be generally perpendicular to the central axis of the cylinder of the flow manifold. The flow manifold may further include a support plate. The support plate may be configured as a shelf at a side of the flow manifold and integrated with a set of the gas ports. The set of the gas ports may include first and second ports configured for sensing a flow rate within the flow manifold.

In some versions, the flow manifold may further include a plurality of mounting posts. A plurality of the mounting posts may be configured on a first side of the main pneumatic path of the flow manifold and a plurality of gas ports may be configured on a second side the main pneumatic path. The second side may be an opposing side of the main pneumatic path relative to the first side. The plurality of mounting posts and the plurality of gas ports may be configured to support the sensor module. The plurality of mounting posts may be configured to support the printed circuit board and the plurality of gas ports may be configured to support a sensor of the plurality of sensors.

The mounting posts may include at least one fixing post configured for receiving a fastener of the sensor module for fixedly coupling the sensor module to the flow manifold. Each of the plurality of mounting posts may have a central axis. The central axes of the mounting posts are substantially parallel. The central axes of the mounting posts may be generally parallel to the central axes of the plurality of gas ports. The inlet of the flow manifold may include a female coupling and the outlet of the pneumatic block assembly may include a male coupling. The inlet of the flow manifold may include a male coupling and the outlet of the pneumatic block assembly may include a female coupling. Optionally, the sensor module assembly may further include a tubular seal. The tubular seal may be configured for mating between the male coupling and the female coupling. The tubular seal may be configured to provide a pneumatic seal between the inlet of the flow manifold and the outlet of the pneumatic block assembly. The tubular seal may be configured for (1) insertion within the female coupling and (2) receiving the male coupling within the tubular seal. The tubular seal may form a multiport seal unit configured to pneumatically seal the plurality of gas ports of the flow manifold with the plurality of sensors of the sensor module. The tubular seal and multiport seal unit may be overmoulded to the flow manifold.

In some versions, the sensor module assembly may further include a face seal, the face seal may be configured for mating between the male coupling and the female coupling. The face seal may be configured to provide a pneumatic seal between the inlet of the flow manifold and the outlet of the pneumatic block assembly.

In some versions, the sensor module assembly may further include a main seal. The main seal may be configured for sealing coupling of the outlet of the flow manifold with the pneumatic path of the housing cover panel. The sensor module assembly may further include a multiport seal unit. The multiport seal unit may be configured to (1) pneumatically seal the plurality of gas ports of the flow manifold with the plurality of sensors of the sensor module, and/or (2) pneumatically seal the outlet of the flow manifold with the pneumatic path of the housing cover of the respiratory therapy apparatus. The multiport seal may include a flow sensor seal and a pressure sensor seal separated by a first seal spacing extension. The multiport seal may include a main seal separated from the flow sensor seal and the pressure sensor seal by a second seal spacing extension. The main seal may be configured for sealing coupling of the outlet of the flow manifold with the pneumatic path of the housing cover panel.

In some versions, the flow manifold may be a moulded component formed of a thermoplastic material. The plurality of sensors may include a pressure sensor and a flow sensor. The plurality of sensors may further include an additional pressure sensor, a microphone or a temperature sensor. In some versions, the pneumatic block assembly may include a housing that includes a volute with a motor and impeller configured to produce air at a positive pressure at the outlet of the pneumatic block.

Some versions of the present technology may include the respiratory therapy apparatus for providing a breathable gas to a patient interface for a respiratory therapy. The respiratory therapy apparatus may include a sensor module assembly having any one or more of the features described herein. The respiratory therapy apparatus may include the pneumatic block assembly. The respiratory therapy apparatus may include an external housing to receive the sensor module assembly and the pneumatic block assembly. The external housing may include the housing cover panel. The sensor module assembly may be configured for seal-based securement within the external housing.

The described methods, systems, devices and apparatus can provide improvements in the technological field of automated apparatus for the treatment of respiratory conditions with breathable gas, including, for example, a ventilator.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Treatment Systems

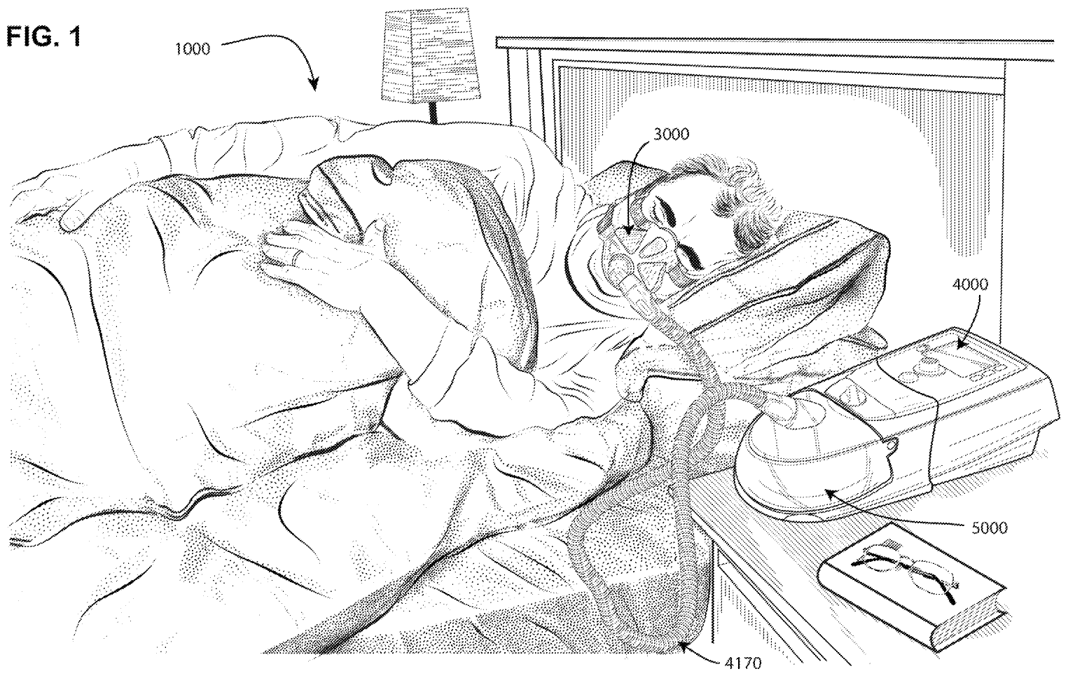

FIG. 1 shows a system including a patient 1000 wearing a patient interface 3000, in the form of a full-face mask, receiving a supply of air at positive pressure from an RPT device 4000, such as a ventilator. Air from the RPT device may be humidified in a humidifier 5000, and passes along an air circuit 4170 to the patient 1000.

### 4.2 Respiratory System and Facial Anatomy

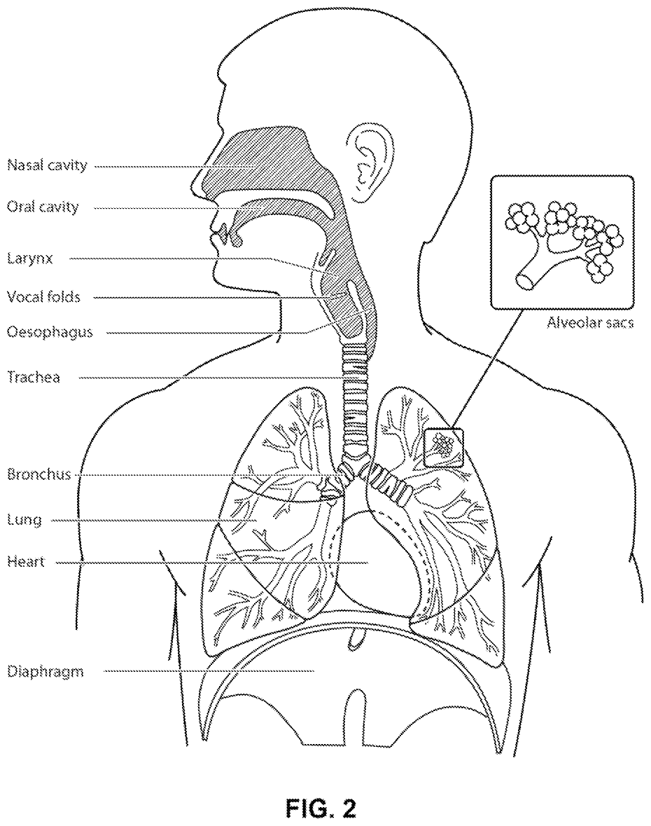

FIG. 2 shows an overview of a human respiratory system including the nasal and oral cavities, the larynx, vocal folds, oesophagus, trachea, bronchus, lung, alveolar sacs, heart and diaphragm.

### 4.3 Patient Interface

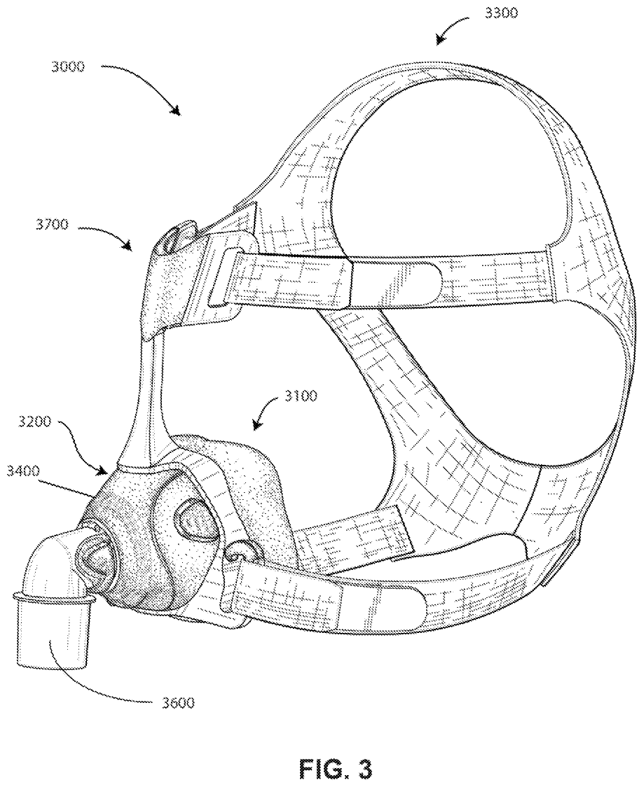

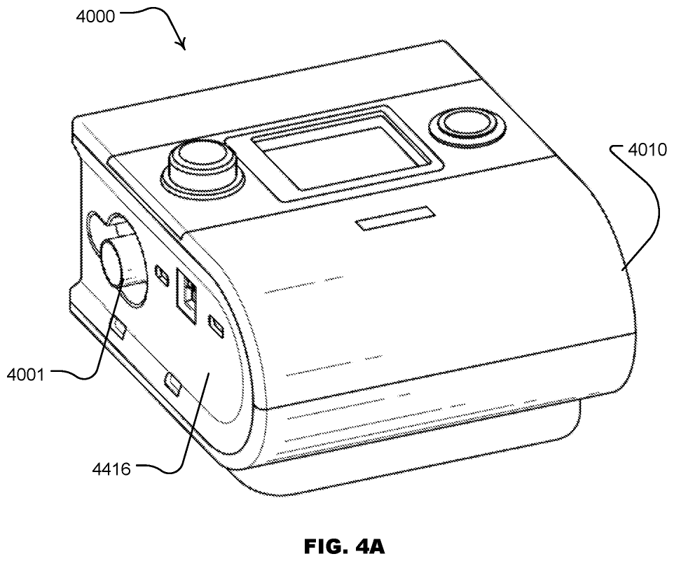

FIG. 3 shows a patient interface in the form of a nasal mask in accordance with one form of the present technology.

### 4.4 Rpt Device

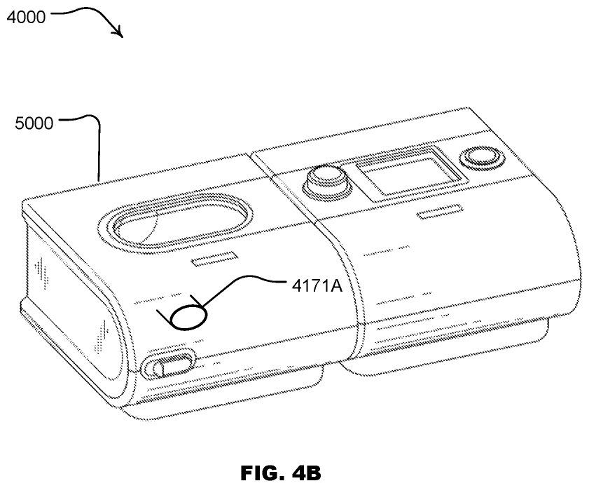

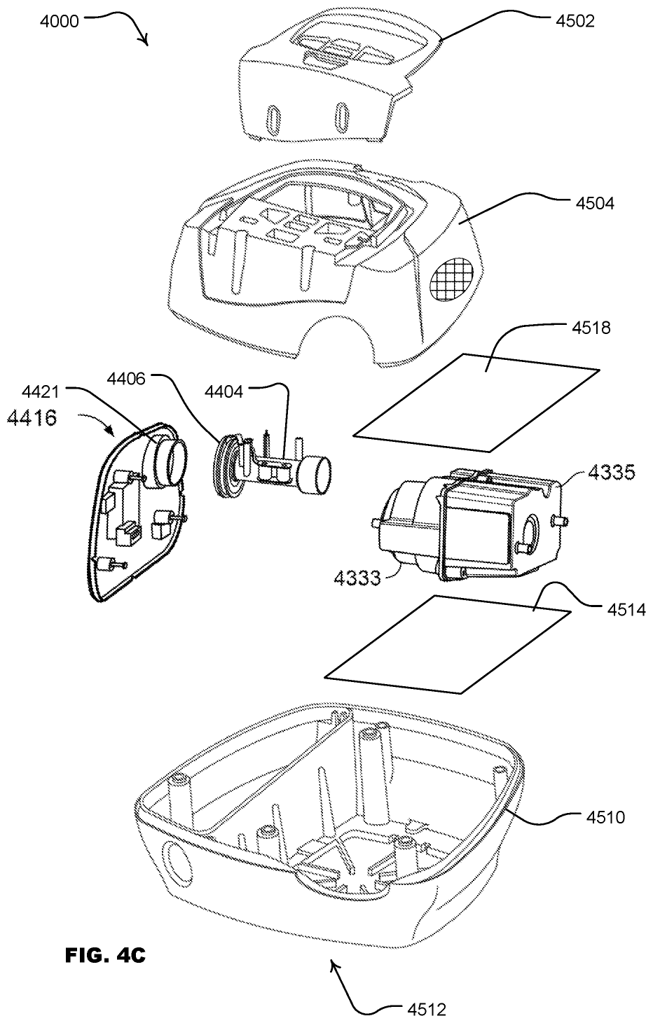

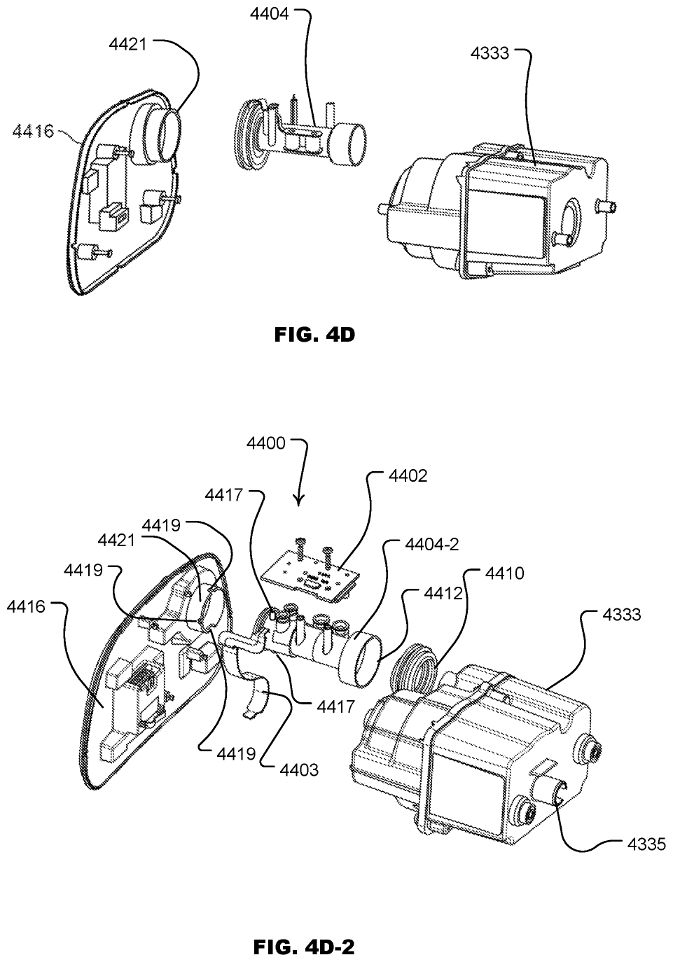

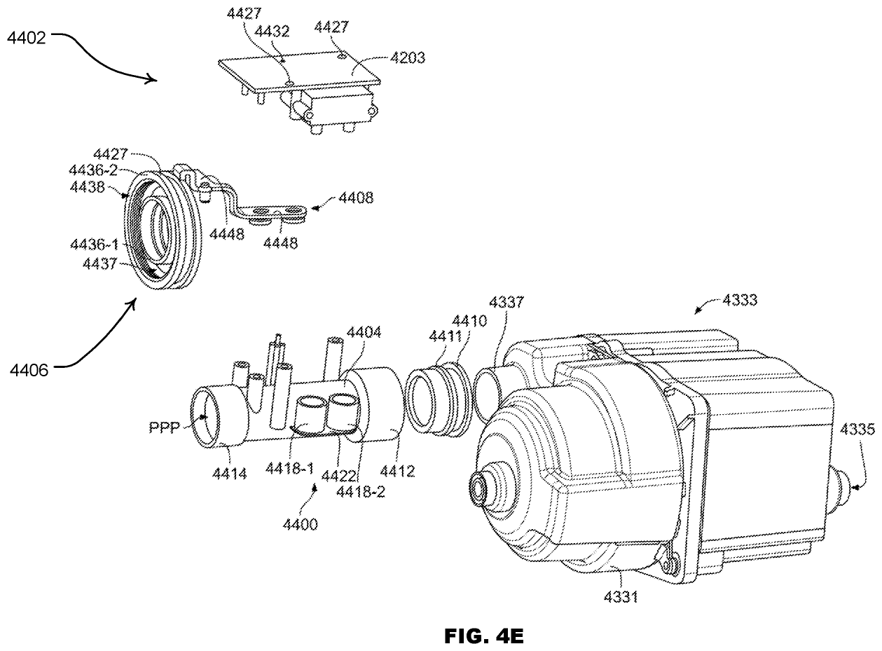

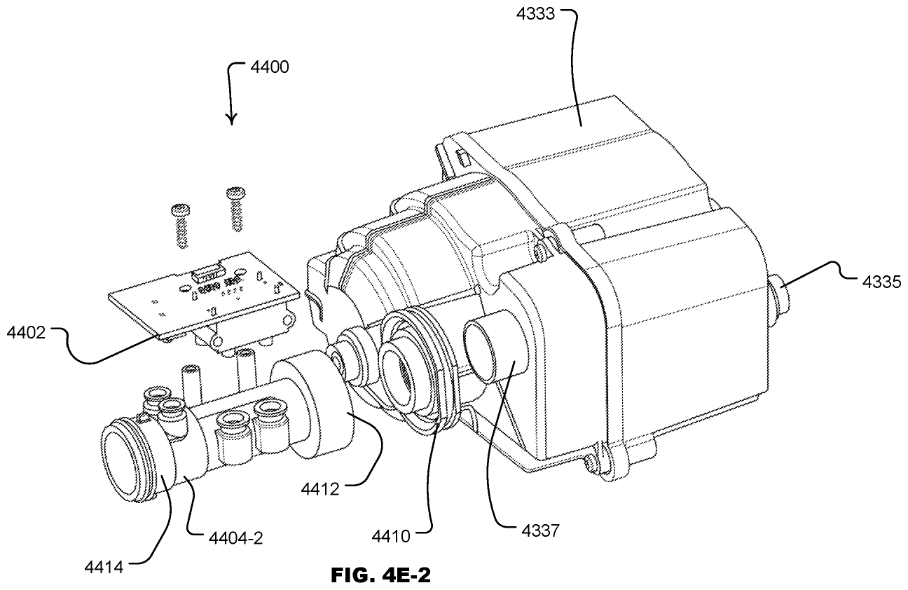

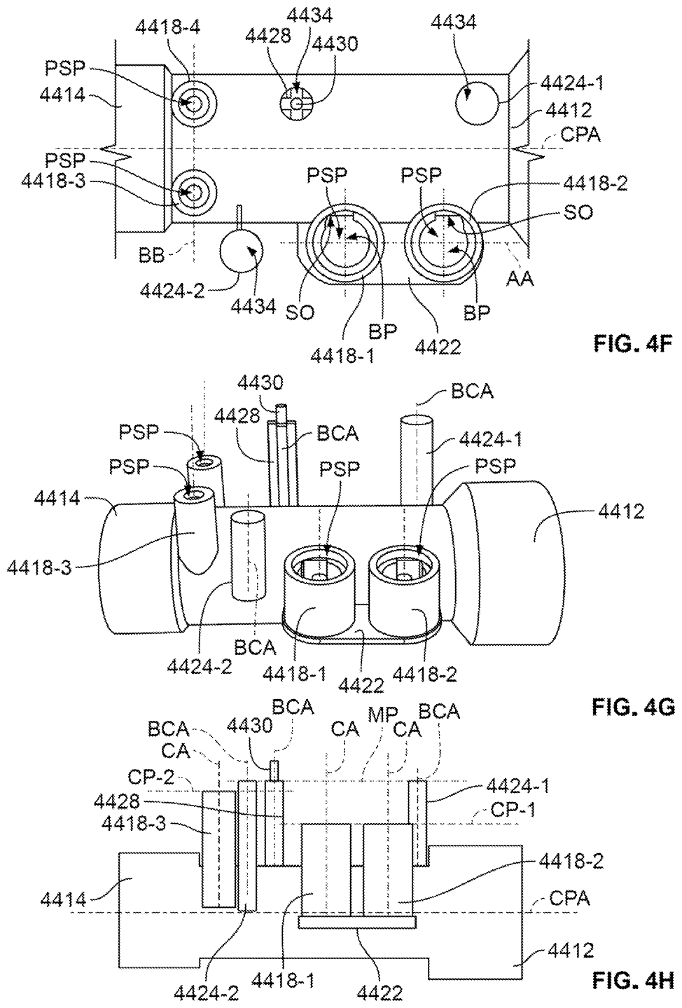

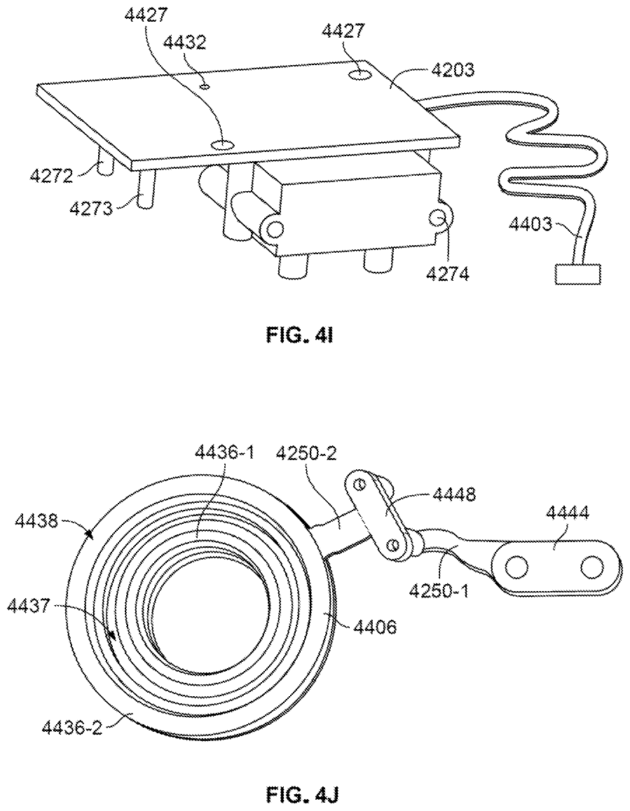

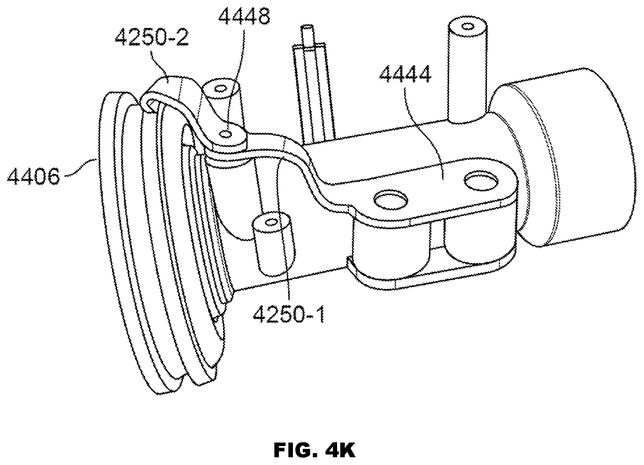

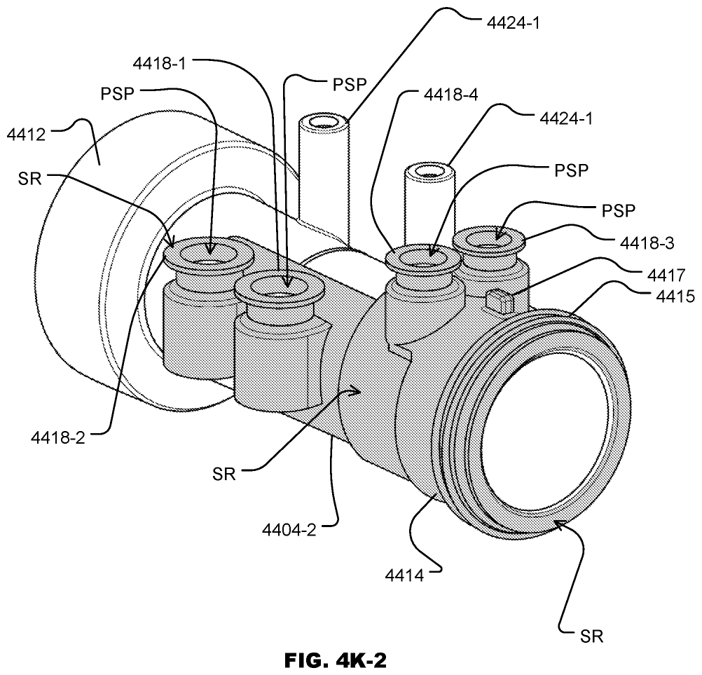

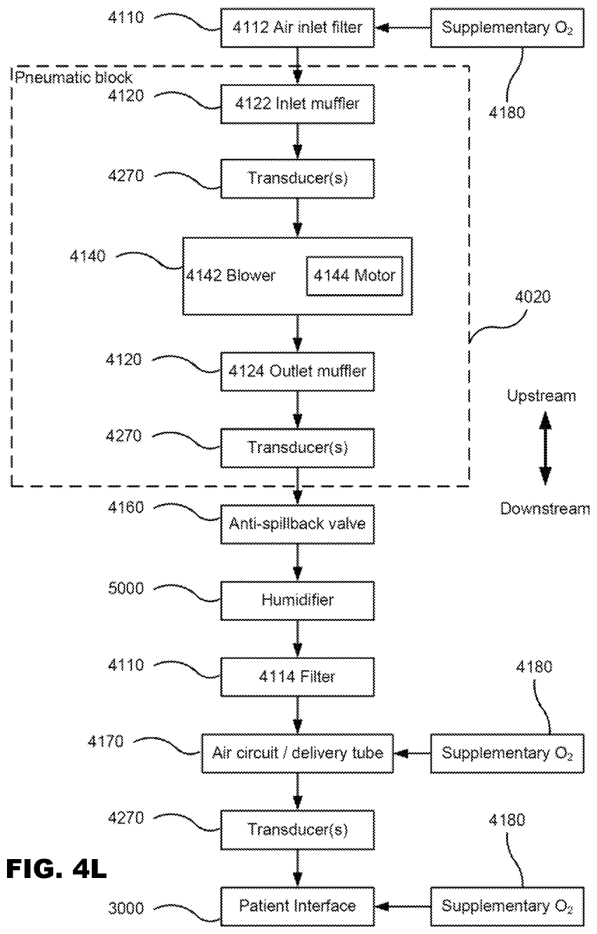

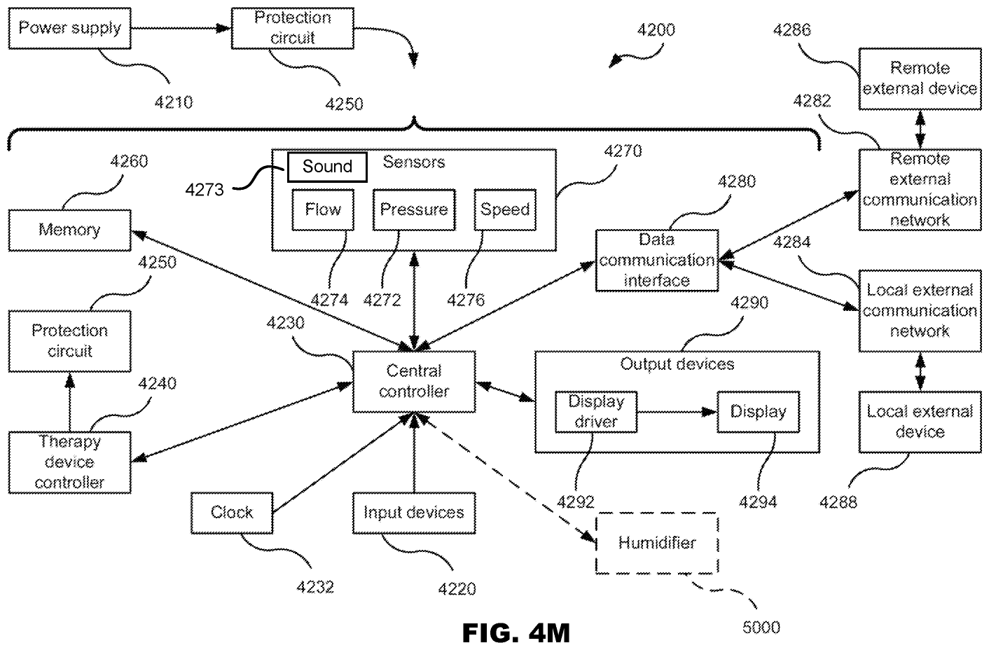

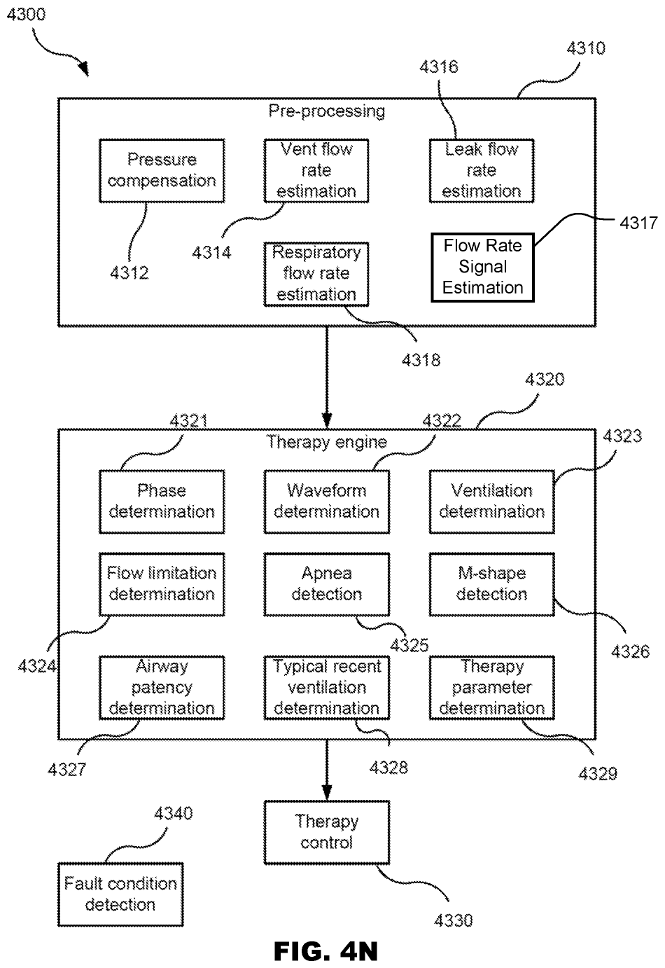

FIG. 4A shows an RPT device, such as a ventilator, in accordance with one form of the present technology.

FIG. 4B shows the RPT device of FIG. 4A with a coupled humidifier 5000, and a portion of a patient circuit such as a respiratory conduit for a mask or endotracheal tube, in accordance with one form of the present technology.

FIG. 4C is an exploded view of example components of the RPT device of FIG. 4A.

FIG. 4D an exploded view of example components of the RPT device of the present technology.

FIG. 4D-2 is an exploded view of example components of another RPT device of the present technology.

FIG. 4E is an exploded view of example components of a sensor module assembly for a pneumatic block of the RPT device of FIG. 4A.

FIG. 4E-2 is an exploded view of example components of a sensor module assembly for a pneumatic block of the RPT device of FIG. 4D-2.

FIG. 4F is a top plan view of a flow manifold of the sensor module assembly of FIG. 4E.

FIG. 4G is an isometric view of the flow manifold of FIG. 4F.

FIG. 4H is a side view of the flow manifold of FIG. 4F.

FIG. 4I shows a sensor module, including sensors and a PCB, of the sensor module assembly of FIG. 4E.

FIG. 4J shows an example seal unit for the sensor module assembly of FIG. 4E.

FIG. 4K shows the seal unit of FIG. 4J assembled with the flow manifold of FIG. 4G.

FIG. 4K-2 shows another implementation of a flow manifold as implemented in FIG. 4D-2 and FIG. 4E-2.

FIG. 4L is a schematic diagram of the pneumatic path of an RPT device in accordance with one form of the present technology. The directions of upstream and downstream are indicated.

FIG. 4M is a schematic diagram of the electrical components of an RPT device in accordance with one form of the present technology.

FIG. 4N is a schematic diagram of the algorithms implemented in an RPT device in accordance with one form of the present technology.

### 4.5 Breathing Waveforms

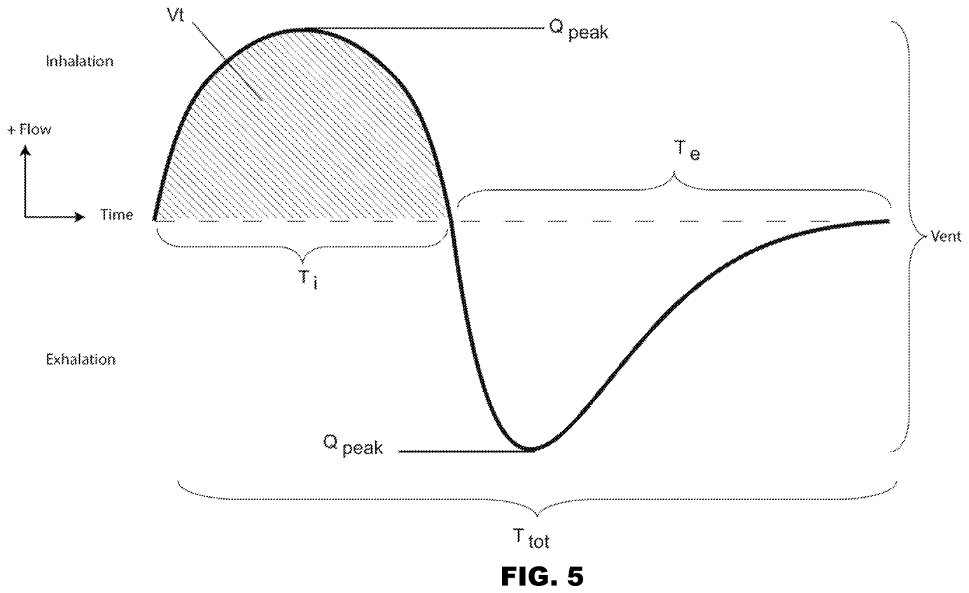

FIG. 5 shows a typical model respiratory flow rate waveform of a person while sleeping. The horizontal axis is time, and the vertical axis is respiratory flow rate. While the parameter values may vary, a typical breath may have the following approximate values: tidal volume, Vt, 0.5 L, inspiratory time, Ti, 1.6 s, peak inspiratory flow rate, Qpeak, 0.4 L/s, expiratory time, Te, 2.4 s, peak expiratory flow rate, Qpeak, −0.5 L/s. The total duration of the breath, Ttot, is about 4 s. The person typically breathes at a rate of about 15 breaths per minute (BPM), with Ventilation, Vent, about 7.5 L/minute. A typical duty cycle, the ratio of Ti to Ttot is about 40%.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising the step of delivering air at a positive pressure, such as Pressure Support, or a high flow rate, of air to the entrance of the airways of a patient 1000.

In certain examples of the present technology, a supply of air at positive pressure or high flow is provided to the nasal passages of the patient via one or both nares.

### 5.2 Treatment Systems

In one form, the present technology comprises an apparatus or device for treating a respiratory disorder. The apparatus or device may comprise an RPT device 4000 for delivering pressurised air to the patient 1000 via an air circuit 4170 to a patient interface 3000. Thus, the RPT device 4000 may be a ventilator. In some versions, the RPT device may be a high flow therapy device that delivers a controlled flow rate of air to the patient through an open patient interface (e.g., a cannula) at rates generally higher than typical inspiration flow rates.

### 5.3 Patient Interface

A non-invasive patient interface 3000 in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to facilitate the delivery of air at positive pressure to the airways. Other patient interface devices may be utilized depending on the type of therapy provided by the RPT.

### 5.4 Rpt Device

An RPT device 4000 in accordance with one aspect of the present technology comprises mechanical and pneumatic components 4100, electrical components 4200 and is configured to execute one or more algorithms 4300. The RPT device may have an external housing 4010, formed in at least two parts. The RPT device 4000 may have a frame or chassis that supports one or more internal components of the RPT device 4000. The RPT device 4000 may include a handle.

The pneumatic path of the RPT device 4000 may comprise one or more air path items, e.g., an inlet air filter 4112, an inlet muffler 4122, a pressure generator 4140 capable of delivering air at positive pressure (e.g., a blower 4142), an outlet muffler 4124 and one or more transducers 4270, such as pressure sensors 4272 and flow rate sensors 4274.

One or more of the air path items may be located within a removable structure which will be referred to as a pneumatic block 4020. The pneumatic block 4020 may be located within the external housing 4010. In one form a pneumatic block 4020 is supported by, or formed as part of a chassis or frame.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Some or all of the electrical components 4200 may be mounted on a single Printed Circuit Board Assembly (PCBA) 4202 such a main PCBA for the central controller. In an alternative form, the RPT device 4000 may include more than one PCBA 4202. For example, a separate sensor related or sensor PCBA 4203 may be included in a sensor module assembly 4400 as described in more detail herein. Such a board can permit greater modularization such as for simple replacement of the sensors without requiring a greater replacement or removal of the other components of a main PCBA. In one example, a plurality of sensors of the sensor PCBA may be configured for sensing gas characteristics of the therapy gas provided by the RPT device 4000 such a near an outlet of a pneumatic block of the RPT device 4000.

An example of an assembly structure of such an RPT device 4000 may be considered in relation to the component illustration of FIGS. 4A, 4B, 4C and 4D. In this regard, FIG. 4A shows an RPT device 4000 showing a display and user interface. An aperture or outlet 4001 at the side cover panel or housing panel 4416, that is pneumatically coupled with the sensor module assembly 4400 and pressure generator as described in more detail herein, may be coupled to a patient circuit 4170 and/or humidifier 5000 (not shown in FIG. 4A) at coupler or adaptor 4171A, which is adapted to couple with a patient circuit 4171. The side cover panel may optionally include an outlet cover infill member that may provide attachment mechanisms for securing the housing of the humidifier 5000 to the housing of the RPT device 4000 as shown in FIG. 4B. Optionally such attachment mechanisms may be provided on the side cover panel without an infill member. Although the humidifier and RPT device 4000 are shown in separable housings in FIG. 4B, in some versions the humidifier 5000 may be an integrated component of the RPT device 4000.

Example components of an assembly of the RPT device of FIG. 4A may be further considered in relation to FIG. 4C showing a configuration of some example components of the RPT device. The RPT device has a UI assembly 4502, which may include the display. The UI assembly may be installed within a top case 4504 of the housing of the RPT device. The top case 4504, which may have as separable end cover (shown as integrated side of top case in FIG. 4C), bottom case 4510 and housing panel 4416 form the outer housing of the RPT device 400. The bottom case 4510 may have a battery door 4512 underneath. The bottom case may support a main PCBA 4202 (not shown). The end cover may include a filter and filter cover. An inlet tube (not shown) may connect the end cover to the pneumatic block 4333. The pneumatic block may be supported by the main frame 4514, which may be considered a chassis. The sensor module assembly 4400 may be between the pneumatic block and the cover panel such as housing panel 4416. The main frame may include a frame cup 4518.

### 5.4.1 RPT Device Mechanical & Pneumatic Components

An RPT device may comprise one or more of the following components in an integral unit. In an alternative form, one or more of the following components may be located as respective separate units. For example, the RPT device may have a pneumatic block 4340 and other assemblies, such as a sensor module assembly 4400, as described in more detail herein in relation to the components of the RPT device 4000 that is illustrated in FIGS. 4A through 4K.

### 5.4.1.1 Air Filter(s)

An RPT device in accordance with one form of the present technology may include an air filter 4110, or a plurality of air filters 4110.

In one form, an inlet air filter 4112 is located at the beginning of the pneumatic path upstream of a pressure generator 4140.

In one form, an outlet air filter 4114, for example an antibacterial filter, is located between an outlet of the pneumatic block 4020 and a patient interface 3000.

### 5.4.1.2 Muffler(s)

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000.

### 5.4.1.3 Pressure Generator

In one form of the present technology, a pressure generator 4140 for delivering a flow, or a supply, of air at positive pressure is a controllable blower 4142. For example, the blower 4142 may include a brushless DC motor 4144 with one or more impellers housed in a volute. The blower may be capable of delivering a supply of air, for example at a rate of up to about 120 litres/minute, at a positive pressure in a range from about 4 cmH₂O to about 20 cmH₂O, or in other forms up to about 30 cmH₂O. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167. In some versions of the present technology, the pressure generator may be configured within the housing of a pneumatic block 4340 as described in more detail herein.

The pressure generator 4140 is under the control of the therapy device controller 4240.

In other forms, a pressure generator 4140 may be a piston-driven pump, a pressure regulator connected to a high-pressure source (e.g. compressed air reservoir), or a bellows.

### 5.4.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 may be constructed and arranged to measure properties such as a flow rate, a pressure or a temperature at that point in the pneumatic path. For example, one or more of the transducers 4270 may be located on a sensor PCBA 4203 that is a part of a sensor module 4402 of the sensor module assembly 4400 as described in more detail herein. Such sensor module 4402, in relation to the assembly, may be configured to measure one or more gas characteristics of the therapy gas traversing within a flow manifold 4404 of the sensor module assembly 4400.

In one form of the present technology, one or more transducers 4270 may be located proximate to the patient interface 3000.

In one form, a signal from a transducer 4270 may be filtered, such as by low-pass, high-pass or band-pass filtering.

### 5.4.1.4.1 Flow Rate Sensor

A flow rate sensor 4274 in accordance with the present technology may be based on a hot wire sensor or a differential pressure transducer, for example, an SDP600 Series differential pressure transducer from SENSIRION. In some versions, the present technology may be implemented without a flow rate sensor, such as where a flow rate estimate signal is generated based on other sensor signals (i.e., not a flow rate sensor signal). The flow rate sensor may be included in the sensor module 4402, such as mounted on the sensor PCBA 4203.

In one form, a signal representing a flow rate such as a total flow rate Qt from the flow rate sensor 4274, and/or an estimate thereof, is received by the central controller 4230.

### 5.4.1.4.2 Sound Sensor

One or more sound sensor(s) 4273 in accordance with the present technology is located in fluid communication with the pneumatic path, and may be included in the sensor module 4402, such as mounted on the sensor PCBA 4203. Thus, the sound sensor 4273 measures the characteristic of the sound within the pneumatic path (e.g., a sound generated by a blower of the RPT or reflected from the patient circuit).

In one form, a signal from the sound sensor 4273 is received by the central controller 4230 for processing.

### 5.4.1.4.3 Pressure Sensor

One or more pressure sensor(s) 4272 in accordance with the present technology is located in fluid communication with the pneumatic path, and may be included in the sensor module 4402, such as mounted on the sensor PCBA 4203. Thus, the pressure sensor 4272 measures the pressure characteristic of the gas within the pneumatic path (e.g., a pressure generated by a blower of the RPT). An example of a suitable pressure transducer is a sensor from the HONEYWELL ASDX series. An alternative suitable pressure transducer is a sensor from the NPA Series from GENERAL ELECTRIC.

In one form, a signal from the pressure sensor 4272 is received by the central controller 4230.

### 5.4.1.4.4 Motor Speed Transducer

In one form of the present technology a motor speed transducer 4276 or sensor is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 5.4.1.4.5 Gas Temperature Sensor

In some versions of the present technology, a gas temperature sensor may be included in the sensor module 4402, such as mounted on the sensor PCBA 4203. In some such implementations, the gas temperature sensor may be a component of another sensor such as a Sensirion SDP-872 flow sensor (e.g., a hot wire sensor) that can generate temperature and flow rate signals but may be an independent temperature sensor or other temperature sensing device. Such a sensor may generate a signal (e.g., electronic) representing measured temperature of gas (air) in, or related to, the gas of the pneumatic path of the RPT. Such a measure may alternatively represent the gas that is ambient to the pneumatic flow path of the RPT. Such a sensor may be located on a PCBA of the RPT, for example. The sensor may generate the sensed temperature in analog and/or digital signals and may be accessed by a processor of the controller 4230 via a sampled signal and/or a memory containing temperature values from such a sensor signal.

### 5.4.1.4.6 Oxygen Sensor

Some versions of the present technology may optionally include one or more oxygen sensors, such as to generate an oxygen sensor signal, adapted to determine an oxygen concentration of gas passing through the pneumatic path of the apparatus such as the RPT. In an implementation, the oxygen concentration of gas passing through respiratory conduit is estimated using an oxygen sensor, which may be included in the sensor module 4402, such as mounted on the sensor PCBA 4203. An oxygen sensor is a device configured to measure oxygen concentration in a gas. Examples of oxygen sensors include, but are not limited to, ultrasonic oxygen sensors, electrical oxygen sensors, chemical oxygen sensors, and optical oxygen sensors. In one implementation, oxygen sensor may be an ultrasonic oxygen sensor that includes an ultrasonic emitter and an ultrasonic receiver.

### 5.4.1.4.7 Other Motor Parameter Sensor(s)

Some versions of the present technology may optionally include one or more sensors or circuit elements for determining or sensing other motor parameters signal(s) such as motor current, motor voltage and/or motor power. For example, one or more sense resistor(s) may be employed to measure currents and/or voltage supplied to the motor of the blower. In some versions, such as with a measured current and a known or measured voltage, an instantaneous power of the motor may be computed (e.g., current×voltage=power) such as with a central controller of the apparatus.

### 5.4.1.5 Anti-Spill Back Valve

In one form of the present technology, an anti-spill back valve is located between the humidifier 5000 and the pneumatic block 4020. The anti-spill back valve is constructed and arranged to reduce the risk that water will flow upstream from the humidifier 5000, for example to the motor 4144.

### 5.4.1.6 Air Circuit

An air circuit 4170 in accordance with an aspect of the present technology is a conduit or a tube constructed and arranged in use to allow a flow of air to travel between two components such as the pneumatic block 4020 and the patient interface 3000.

In particular, the air circuit 4170 may be in fluid connection with the outlet of the pneumatic block and the patient interface. The air circuit may be referred to as an air delivery tube. In some cases there may be separate limbs of the circuit for inspiration and expiration. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller 4230 or a humidifier controller 5250. One example of an air circuit 4170 comprising a heated wire circuit is described in United States Patent Application No. US/2011/0023874, which is incorporated herewithin in its entirety by reference.

### 5.4.1.7 Oxygen Delivery

In one form of the present technology, supplemental oxygen 4180 is delivered to one or more points in the pneumatic path, such as upstream of the pneumatic block 4020, to the air circuit 4170 and/or to the patient interface 3000.

### 5.4.1.8 Example Pneumatic Assemblies

Some RPT devices 4000 of the present technology may be implemented with separable pneumatic assemblies to simplify manufacture, removal, maintenance and/or replacement of discrete parts of the RPT device. Some such pneumatic assemblies may include a pneumatic block 4340 and a sensor module assembly 4400. These devices may be readily separated, such as for repair of one or the other, and/or removable from a housing of the RPT device 4000. Components of these devices are described in more detail herein.

For example, future problems with the sensors or the sensor PCBA can be easily addressed and diagnosed by removing the sensor module assembly whole and easily unscrewing the sensor PCBA and replacing it or diagnosing issues with it. This can improve servicing and can also simplify installation and testing of the sensor module assembly separate from a central controller and/or pneumatic block of the RPT device.

### 5.4.1.8.1 Pneumatic Block 4340

Such a pneumatic block 4340, as previously described may house one or more blowers of the pressure generator 4140. Thus, the pneumatic block 4333 will have a housing 4331 that itself can be inserted into an external housing of the RPT device 4000 as described in more detail herein. The housing 4331 of the pneumatic block will house the volute of the blower(s) and the motor(s) for the blower(s). The housing 4331 of the pneumatic block 4333 may have an inlet 4335 such as to supply air to the blower for pressurization and an outlet 4337 that serves to provide the breathable gas pressurized by the blower(s). The pneumatic block 4333 may also optionally include a breathable gas inlet control device, or non-return valve, such as the device described in International Patent Application No. PCT/AU2011/000341, the entire disclosure of which is incorporated herein by reference. Such a device may typically include an aperture in an inlet chamber that may be sealed by an inlet flow seal. Controlled movement of the inlet flow seal serves to impede flow by preventing or permitting gas transfer between an anterior portion of the gas inlet chamber and an exterior of the seal and a posterior portion of the gas inlet interior of the seal. The inlet flow seal is coupled to a seal activation chamber that is pneumatically operated with different pressure conditions for controlling the seal. In some versions, the pneumatic block may include any one or more of acoustic chambers, inertance tubes, acoustic impedance tubes, muffling foams, heat dissipation material. Examples of such a pneumatic block may be considered in relation to the disclosure of Australian Provisional Patent Application No. 2019903508, filed on 20 Sep. 2019, the entire disclosure of which is incorporated herein by reference.

### 5.4.1.8.2 Sensor Module Assembly 4400

As previously mentioned, some versions of the RPT device 4000 of the present technology may include a sensor module assembly 4400. Such an assembly may be designed for simplifying replacement of the sensor components of the RPT device 4000, such as potentially without replacement and/or removal of the pneumatic block from the RPT device 4000. In this regard, the sensor module assembly 4400 is designed to be readily removable from the pneumatic block. As illustrated in the figures (e.g., FIG. 4E), the sensor module assembly 4400 may include a sensor module 4402, a flow manifold 4404, and one or more seals (e.g., main seal 4406, sensor seal 4408 and/or block coupling seal 4410). When coupled together, these components form the sensor module assembly 4400. These assembly components may be considered in relation to FIGS. 4F through 4J. Although the figures generally illustrate the sensor module assembly between the pneumatic block and a housing panel, it will be understood that the sensor module assembly may be implemented in other assemblies such that it serves its sensing and pneumatic functions. In this regard, in other assemblies it may be coupled/installed in the pneumatic path after a blower or pneumatic block and before a humidifier when present. Moreover, it may be applied to such assemblies with other components or pneumatic couplings (e.g., tubes) suitable for providing the pneumatic connections. Preferably, such connections and assemblies are configured to simplify removal and installation. In this regard, the pneumatic couplings may serve as the structure for retaining/securing the sensor module assembly within the housing of the RPT device, and it may do so without additional fasteners coupling that assembly with the housing structures of the RPT device (e.g., without fastening it to a chassis with fasteners).

In the examples, a primary pneumatic component of the sensor module assembly 4400 is the flow manifold 4404. The flow manifold conducts the therapy gas between the patient circuit 4170 and the pneumatic block 4340. In the illustrated example, the flow manifold 4404 serves as a breathable gas (e.g., therapy gas) passage, or pneumatic path, from the pneumatic block (e.g., the pressure generator). The flow manifold may be comprised of a cylindrical structure. In the illustrated example, the main or primary pneumatic path PPP may pass through the flow manifold without any substantial bend via the cylindrical portion. Thus, the primary pneumatic path may be relatively straight through the cylindrical portion CP of flow manifold 4404 such as generally parallel to a central path axis CPA (imaginary). In this regard, as illustrated in FIG. 4H, the cylindrical portion CP of flow manifold 4404 may be defined by a central path axis CPA (imaginary) through the cylinder of the cylindrical portion of the flow manifold 4404 (e.g., from end to end). A central cylindrical portion CP may be between one or more coupling ends 4412, 4414 of the flow manifold 4404. Thus, the flow manifold may have a first coupling end 4412 for pneumatic coupling with an outlet 4337 of the pneumatic block 4333. Moreover, the flow manifold may have a second coupling end 4414 for pneumatic coupling with an outlet 4001 of a housing panel 4416 of the RPT device 4000 that may serve as an exterior housing panel of the RPT device 4000 (see, e.g., FIG. 4C).

The flow manifold includes one or more gas ports 4418. As best seen in the illustration of the example of FIGS. 4E and 4F, each of a plurality of the gas ports (4418-1, 4418-2, 4418-3, 4418-4) can provide a pneumatic sensing passage PSP from the main or primary pneumatic path of the cylindrical portion of the flow manifold to a sensing transducer of a sensor of the sensor module 4402 of the sensor module assembly 4400. In this regard, any of the previously described sensors may sense a gas characteristic with one or more of the gas ports. As illustrated in FIGS. 4E and 4G, any one or more of the gas ports may be formed with an extension to conduct the gas from the main pneumatic path of the flow manifold to a sensing transducer of a sensor of the module. As illustrated in FIGS. 4F and 4G, the gas port extensions may be cylindrical at an internal and/or external surface. Thus, each cylindrical extension may be defined by a central axis CA (imaginary) along a cylinder portion of the gas port extension. In some versions, such axes are generally parallel to each other. Moreover, such axes CA may also be generally perpendicular to the central path axis CPA through the cylinder of the cylindrical portion of the flow manifold 4404.

In some cases, each of a plurality of such extensions (e.g., some or all) may optionally be formed with a height that is raised above the cylindrical portion of the flow manifold. In some such versions, the heights may end at a plane CP (e.g., CP-1 and/or CP-2 in FIG. 4H). Such a common peak of the extensions may facilitate their coupling with sensors that are mounted to a single PCBA.

In some such versions, the flow manifold may include a gas port 4418-1 and/or a gas port 4418-2 such as for flow sensing. In this regard, a gas port 4418-1 and gas port 4418-2 may be coupled to a flow rate sensor of the sensor module 4402. As illustrated in FIG. 4F, a central portion of each extension of the gas ports 4418-1 and 4418-2 forms the pneumatic sensing passage PSP or pneumatic channel of the gas port. As further illustrated in FIG. 4F, the gas ports 4418-1 and 4418-2 are offset from the central path axis CPA through the cylinder of the cylindrical portion of the flow manifold. Each of these channel ends at a base portion BP. A side opening SO within each the channel then provides fluid access to the primary pneumatic path of the cylindrical portion of the flow manifold. The gas port 4418-1 and gas port 4418-2 may be integrated with a support plate 4422 that forms an integrated shelf that provides support for the set of ports at the base portion BP. The support plate 4422 may be a projection of the material of the flow manifold at a side of the cylindrical portion of the flow manifold.

In the illustrated version of FIG. 4F, the gas port 4418-1 and gas port 4418-2 are both offset to the same side of the cylinder of the cylindrical portion of the flow manifold. Moreover, their alignment is generally parallel to the gas flow of the flow manifold through its main pneumatic path. Thus, a set of the gas ports may be arranged in a series along a length of a side of the cylindrical portion of the flow manifold. In this regard, as illustrated in FIG. 4F, a line AA (imaginary) running through a center point of each of the gas port 4418-1 and gas port 4418-2 forms a generally parallel line relative to the central path axis CPA through the cylinder of the cylindrical portion of the flow manifold. Such a parallel flow alignment of the two ports can improve sensing along the primary pneumatic path as it changes between the two side openings SO of the channels of the gas ports 4418-1 and 4418-2. Such an alignment can help to detect a pressure differential between the side openings SO. In the illustrated version, the height of each of the gas port 4418-1 and gas port 4418-2 rise to a common plane CP-1.

In some versions, the flow manifold may include a gas port 4418-3 and/or a gas port 4418-4 such as for pressure sensing and/or sound sensing such as with a microphone type sensor. For example, a gas port 4418-3 and gas port 4418-4 may one or both of the ports may be coupled to a pressure sensor(s) of the sensor module 4402 or any of the other sensors. The gas ports 4418-3 and 4418-4 extend out of a top half of the cylindrical portion of the flow manifold. As illustrated in FIG. 4F, a central portion of each extension of the gas ports 4418-3 and 4418-4 forms the pneumatic sensing passage PSP or pneumatic channel of the gas port. As further illustrated in FIG. 4F, the gas ports 4418-3 and 4418-4 are offset from the central path axis CPA through the cylinder of the cylindrical portion of the flow manifold but at opposing sides of the central path axis. Nevertheless, the pneumatic sensing passage PSP or pneumatic channel of the gas port from the peak opens directly into the primary pneumatic path of the cylindrical portion of the flow manifold without a tortuous path (i.e., straight). Thus, no side opening within the extension of these gas ports is necessary.

Optionally, the gas port 4418-3 and gas port 4418-4 may be aligned to be generally perpendicular to the gas flow of the flow manifold through its main pneumatic path. In this regard, as illustrated in FIG. 4F, a line BB (imaginary) running through a center point of each of the gas port 4418-3 and gas port 4418-4 forms a generally perpendicular line relative to the central path axis CPA through the cylinder of the cylindrical portion of the flow manifold. Such a perpendicular flow alignment of the two ports can permit sensing of a more uniform condition of the gas characteristic within or along the primary pneumatic path despite the flow within or along the path the primary pneumatic path. In the illustrated version, the height of each of the gas port 4418-3 and gas port 4418-4 rise to a common plane CP-2.

The flow manifold may also include one or more mounting structures for affixing sensor module 4402 to the flow manifold 4404. Such mounting structures may be at least in part integrated with the moulded structure of the flow manifold. Such mounting structures may be affixed to a sensor PCBA such as with one or more fastener(s). For example, as illustrated in FIGS. 4F and 4G, the flow manifold may include one or more (e.g., a plurality of) fixing posts 4424-1, 4424-2 such as screw bosses. For example, one or more posts may be integrated with the flow manifold. In some versions, such fixing posts may have an aperture at a peak 4426 of the fixing post such as to receive a fixing member (e.g., a screw or other fastener). Optionally, the sensor PCBA may have fastener apertures 4427 for applying such fasteners.

As further illustrated in FIG. 4F the mounting structures may include, for example, an alignment post 4428. Such an alignment post may include a pin 4430, such as for mounting the sensor PCBA in a corresponding aperture 4432 of the sensor PCBA. Alternatively, a pin 4430 of the sensor PCBA may be inserted into an aperture at a peak of the alignment post. Such mounting posts (fixing or alignment) may then be employed for securing the sensor PCBA with the flow manifold using one or more fasteners. Such securement thereby aligns the sensor transducers with the gas ports for the sensing as previously described. In some versions, the mounting posts may be cylindrical or tee structures as illustrated in FIGS. 4F and 4G. The mounting posts may each be defined by a central axis BCA running from its connection to the flow manifold cylindrical portion to a peak of the mounting boss. Such central axes of the mounting bosses may be generally parallel to each other. Moreover, the central axes BCA of the mounting posts may be generally parallel to the central axes CA of the extensions of any one, more or all of the gas ports 4418-1, 4418-2, 4418-3 and 4418-4. As illustrated in FIG. 4H, the mounting/fixing posts may rise to a height that peaks at a common mounting plane MP to receive a surface of the sensor PCBA. Thus, the mounting/fixing posts may each have a peak surface 4434 for contact with a surface of the sensor PCBA at the common mounting plane MP.

As illustrated in FIGS. 4E, 4J and 4K, the sensor module assembly 4400 may include and/or utilize one or more seals for pneumatic sealing so as to enable operations within the RPT device 4000. The seals, which may be formed with a flexible compliant material, such as a rubber or silicone material for example, are not only important for sealing the air path but also serve as compliant interfaces that improve removability and installation. This compliance helps to ensure a reliable module that can easily be assembled within tight spacing as the outlet panel cover slides on during assembly and the sliding of the cover panel plies a surface of the panel against a seal for aligning the outlet of the panel with the flow manifold and thereby effecting the seal and also effecting the securement of the sensor module assembly 4400 in position between the panel outlet and the outlet of the pneumatic block. In this regard, this seal-based securement of the sensor manifold assembly 4400 for use within the RPT device 4000 may be achieved without otherwise applying (or requiring) other fasteners, such as hardware fasteners (e.g., screws, bolts, etc.), that affix the sensor manifold assembly 4400 to a housing structure such a chassis of the RPT device 4000. In this way, it is substantially secured for use within the housing by its pneumatic couplings, which thereby prevents internal movement during use. In this regard, the sensor manifold assembly 4400 may be retained in the housing by the pneumatic connection of its seals with the cover panel, or other housing structure or such a housing structure, panel or chassis with a pneumatic pathway, and/or the pneumatic block or an outlet path thereof. In this regard, the sensor module assembly, including its sensors and a PCB for such sensors, may be secured (e.g., preventing internal shifting) within the housing of the RPT device substantially by its pneumatic connections or seals. Thus, such securement does not require additional fixing components (e.g., screws or fasteners that might require tools) to secure it to, or within, the housing. As such, an easy installation, removal and/or replacement is facilitated.

Thus, a main seal 4406 may be utilized by the assembly for pneumatic coupling between the second coupling end 4414 so as to seal the second coupling end 4414 with an outlet of a housing panel 4416. The main seal 4406 may comprise multiple rims 4436 such as a first rim 4436-1 and a second rim 4436-2, that can assist with improving the quality of the sealing. The rims may be annular. In the illustrated example, the first rim 4436-1 may be an annular ring inside an annular ring of second rim 4436-2. Thus, a diameter associated with a surface of the second rim 4436-2 may be larger than a diameter associated with an inside surface of the second rim 4436-2. As illustrated in FIG. 4E, the two rims form an annular space or cavity 4437 between these surfaces. In the example, the first rim 4436-1 may be sized to be snuggly fitted about the second coupling end 4414 as a sleeve to provide a seal against the outer surface diameter of the second coupling end 4414 of the flow manifold 4404. Moreover, the second rim 4436-2 which one or more of its seal surface(s) 4438 when plied by the cover panel serves a buttress for the sensor manifold assembly 4400 when the cover panel is plied into its closed position on the housing of the RPT device 4000. Moreover, the diameter of the inside surface of the second rim 4436-2 may be sized so that it circumscribes the outlet 4440 of the cover panel (housing panel 4416) to permit sealing the connection between the coupling end of the flow manifold and the outlet 4440. As illustrated in FIG. 4C, the cover panel may optionally have an annular nipple 4442 about a pneumatic path of the outlet 4440 which may be sized to fit within the annular cavity 4437 of the main seal. Such a nipple can help to improve the sealing as well as simplify the alignment of the connection of the flow manifold.

On the opposite side of the flow manifold, (i.e., the first coupling end 4412), the flow manifold of the sensor module assembly may be pneumatically coupled to the pneumatic block 4333 by utilizing the block coupling seal 4410. Such a seal may be tubular. Such a tubular seal may be configured for mating between a male coupling and a female coupling to provide a pneumatic seal between an inlet of the flow manifold and an outlet of the pneumatic block assembly. For example, as illustrated in the example of FIG. 4E, the block coupling seal 4410 is sized to be snuggly fitted about an outlet rim of the outlet 4337 of the pneumatic block 4333 that may be formed as a male coupling. Thus, the cylindrical inner surface of the block coupling seal 4410 seals against an outer cylindrical surface of the outlet rim of the outlet 4337. Moreover, the cylindrical outer surface of the block coupling seal 4410 seals against an inner cylindrical surface of the first coupling end 4412 (e.g., a female coupling) of the flow manifold 4404. Optionally, a surface of the block coupling seal may include one or more coupling ridges 4411. Such ridges may be annular about the surface and can provide improved securement of the pneumatic connection. While some embodiments utilize such a tubular seal, in some versions, the seal 4410 may be a face seal between the opposing couplings.

The sensor module assembly may include one or more sensor seals 4408. For example, a flow sensor seal 4444 may have one or more apertures (e.g., two) for sealing the transducer(s) of the flow rate sensor with the pneumatic sensing paths (PSP) of the gas port(s) of the flow manifold that may be associated with flow rate sensing. Similarly, a pressure sensor seal 4448 may have one or more apertures (e.g., two) for sealing the transducer(s) of other sensors such as the pressure sensor(s) and/or sound sensor(s) with the pneumatic sensing paths (PSP) of the gas port(s) of the flow manifold that may be associated with pressure and/or sound sensing.

In some versions such seals may be configured as a multiport seal unit. For example, such sensor seals may be moulded so as to be integrated with one or more seal spacing extensions 4450-1, 4450-2 that integrate multiple seals into a singular sealing unit. Thus, a first seal spacing extension 4450-1 may join the flow sensor seal 4444 to the pressure sensor seal 4448. Moreover, a second seal spacing extension 4450-2 may join the pressure sensor seal 4448 with the main seal 4406. In the example, the main seal and the flow sensor seal are joined by the pressure sensor seal and the seal spacing extensions on opposing sides of the pressure sensor seal. Such seal spacing extensions can simplify manufacture and assembly or re-assembly when repair or replacement to the sensor module is necessary.

Another implementation of the sensor module assembly may be considered in relation to the illustrations of FIGS. 4D-2, 4E-2 and 4K-2. The components of these figures are similar to the components of FIGS. 4D, 4E and 4G as previously described and as such have similar reference numbers. However, the flow manifold 4404-2 of 4K-2 may be implemented with some differences. For example, flow manifold 4404-2 may have one or more alignment tab(s) 4417. The alignment tabs permit a consistent installation orientation of the flow manifold when the alignment tables are aligned with one or more tab recess(es) 4419 that may be included on the outlet port of the housing panel 4416 that leads to the aperture of outlet 4001. As illustrated in FIG. 4D-2, the outlet port may extend into the internal portion of the housing of the RPT device with an extension 4421 from the housing panel 4416. In the version of FIG. 4D-2, the second coupling end 4414 of the flow manifold includes one or more coupling end seal(s) 4415 (see FIG. 4K-2) such as to permit insertion of the second coupling end 4414 into the extension 4421 of the housing panel 4416. The seals 4415 permit pneumatic sealing of such a coupling. In such an implementation, the more substantial main seal 4406 as shown in FIG. 4J or 4K at the second coupling end 4414 is not required. The opposite side of the flow manifold 4404-2 may still utilize a block coupling seal 4410 at the first coupling end 4412 of the flow manifold 4404-2 as previously described and shown in FIGS. 4D-2 and 4E-2. Optionally, the block coupling seal 4410 may then be integrated with additional seal structures such as any one or more of the flow sensor seal 4444 and/or pressure sensor seal 4448 and/or the seal spacing extension 4450 as previously described in relation to the main seal 4406. For example, as illustrated in FIG. 4K-2, the seals associated with the gas ports and second coupling end 4414 may be overmoulded to the structure of the flow manifold such that the seal and flow manifold become an integrated unit. Such an overmoulding essentially encapsulates seal material (e.g., silicone) over a portion of the flow manifold in the vicinities of sealing and may simplify installation, replacement and assembly. Such encapsulation is illustrated in FIG. 4K-2 by the shaded region SR.

In the version of the flow manifold of FIG. 4K-2, two fixing posts 4424-1 are implemented on one side of the flow channel (main pneumatic path) of the flow manifold. These posts may be implemented as screw bosses, for example. The gas port 4418-1 and gas port 4418-2 that may be coupled to a flow rate sensor of the sensor module 4402 may be integrated at an opposing side of the flow channel or main pneumatic path, opposite the fixing posts. Such orientation can permit a more balanced support of the sensor module when assembled to the flow manifold.

### 5.4.2 RPT Device Electrical Components

### 5.4.2.1 Power Supply

A power supply 4210 may be located internal or external of the external housing 4010 of the RPT device 4000.

In one form of the present technology, power supply 4210 provides electrical power to the RPT device 4000 only. In another form of the present technology, power supply 4210 provides electrical power to both RPT device 4000 and humidifier 5000.

### 5.4.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form, the input device 4220 may be constructed and arranged to allow a person to select a value and/or a menu option.

### 5.4.2.3 Central Controller

In one form of the present technology, the central controller 4230 is one or a plurality of processors suitable to control an RPT device 4000.

Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 4230 is a dedicated electronic circuit.

In one form, the central controller 4230 is an application-specific integrated circuit. In another form, the central controller 4230 comprises discrete electronic components.

The central controller 4230 may be configured to receive input signal(s) from one or more transducers 4270, and one or more input devices 4220.

The central controller 4230 may be configured to provide output signal(s) to one or more of an output device 4290, a therapy device controller 4240, a data communication interface 4280 and humidifier controller 5250.

In some forms of the present technology, the central controller 4230 is configured to implement the one or more methodologies described herein, such as the one or more algorithms 4300 expressed as computer programs stored in a non-transitory computer readable storage medium, such as memory 4260. In some forms of the present technology, the central controller 4230 may be integrated with an RPT device 4000. However, in some forms of the present technology, some methodologies may be performed by a remotely located device. For example, the remotely located device may determine control settings for a ventilator or detect respiratory related events by analysis of stored data such as from any of the sensors described herein.

### 5.4.2.4 Clock

The RPT device 4000 may include a clock 4232 that is connected to the central controller 4230.

### 5.4.2.5 Therapy Device Controller

In one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 5.4.2.6 Protection Circuits

The one or more protection circuits 4250 in accordance with the present technology may comprise an electrical protection circuit, a temperature and/or pressure safety circuit.

### 5.4.2.7 Memory

In accordance with one form of the present technology the RPT device 4000 includes memory 4260, e.g., non-volatile memory. In some forms, memory 4260 may include battery powered static RAM. In some forms, memory 4260 may include volatile RAM.

Memory 4260 may be located on the PCBA 4202. Memory 4260 may be in the form of EEPROM, or NAND flash.

Additionally or alternatively, RPT device 4000 includes a removable form of memory 4260, for example a memory card made in accordance with the Secure Digital (SD) standard.

In one form of the present technology, the memory 4260 acts as a non-transitory computer readable storage medium on which is stored computer program instructions expressing the one or more methodologies described herein, such as the one or more algorithms 4300 including, for example, the methodologies described in relation to flow rate estimation described in more detail herein. The memory 4260 may also act as a volatile or non-volatile storage medium for data acquired, collected, used or generated as one or more of the methodologies described herein are executed as instructions by one or more processors.

Optionally, in some forms of the present technology, the sensor PCBA 4203 may include such a non-volatile memory. Such a memory may include calibration data for the one or more sensors of the sensor module 4402. This data record, in addition to the isolation of the sensors as part of the replaceable sensor module assembly 4400 as described herein, can permit pre-calibration at manufacture/assembly time so that such calibration does not need to be performed in the field such as in relation to an onsite replacement (e.g., home or hospital) of the assembly where the RPT device 4000 is used or serviced.

### 5.4.2.8 Sensor Module Interface

As previously described, the sensor module assembly, including the sensor module is an easily replaceable component of the RPT device. As such, the main PCBA of the main controller (central controller 4230) may include an interface (wire harness or bus) for powering, polling with, and/or receiving data from the sensors and/or memory of the sensor module 4402. For example, establishing, or removing, such electrical connection may be made by a wired connector 4403 or wire harness that may be coupled to a corresponding connector of the main PCBA. Thus, the sensor module 4402 may include one or more components for such a removeable electrical connection (not shown) to the main PCBA.

### 5.4.2.9 Data Communication Systems

In one form of the present technology, a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g. via Ethernet, or optical fibre) or a wireless protocol (e.g. CDMA, GSM, LTE) to connect to the Internet.

In one form, local external communication network 4284 utilises one or more communication standards, such as Bluetooth, or a consumer infrared protocol.

In one form, remote external device 4286 is one or more computers, for example a cluster of networked computers. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet or remote control.

### 5.4.2.10 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 5.4.2.10.1 Display Driver

A display driver 4292 receives as an input the characters, symbols, or images intended for display on the display 4294, and converts them to commands that cause the display 4294 to display those characters, symbols, or images.

### 5.4.2.10.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

### 5.4.3 RPT Device Algorithms

### 5.4.3.1 Pre-Processing Module

A pre-processing module 4310 in accordance with one form of the present technology receives as an input a signal from a transducer 4270, for example a flow rate sensor 4274 or pressure sensor 4272, and performs one or more process steps to calculate one or more output values that will be used as an input to another module, for example a therapy engine module 4320.

In one form of the present technology, the output values include the interface or mask pressure Pm, the respiratory flow rate Qr, and the leak flow rate Ql.

In various forms of the present technology, the pre-processing module 4310 comprises one or more of the following algorithms: pressure compensation 4312, vent flow rate estimation 4314, leak flow rate estimation 4316, flow rate signal estimation 4317 and respiratory flow rate estimation 4318.

### 5.4.3.1.1 Pressure Compensation

In one form of the present technology, a pressure compensation algorithm 4312 receives as an input a signal indicative of the pressure in the pneumatic path proximal to an outlet of the pneumatic block. The pressure compensation algorithm 4312 estimates the pressure drop through the air circuit 4170 and provides as an output an estimated pressure, Pm, in the patient interface 3000.

### 5.4.3.1.2 Vent Flow Rate Estimation

In one form of the present technology, a vent flow rate estimation algorithm 4314 receives as an input an estimated pressure, Pm, in the patient interface 3000 and estimates a vent flow rate of air, Qv, from a vent 3400 in a patient interface 3000.

### 5.4.3.1.3 Leak Flow Rate Estimation

In one form of the present technology, a leak flow rate estimation algorithm 4316 receives as an input a total flow rate, Qt, and a vent flow rate Qv, and provides as an output an estimate Ql of the leak flow rate. In one form, the leak flow rate estimation algorithm 4316 estimates the leak flow rate Ql by calculating an average of the difference between total flow rate Qt and vent flow rate Qv over a period sufficiently long to include several breathing cycles, e.g. about 10 seconds.

In one form, the leak flow rate estimation algorithm 4316 receives as an input a total flow rate Qt, a vent flow rate Qv, and an estimated pressure, Pm, in the patient interface 3000, and provides as an output a leak flow rate Ql, by calculating a leak conductance, and determining a leak flow rate Ql to be a function of leak conductance and pressure, Pm. Leak conductance is calculated as the quotient of low pass filtered non-vent flow rate equal to the difference between total flow rate Qt and vent flow rate Qv, and low pass filtered square root of pressure Pm, where the low pass filter time constant has a value sufficiently long to include several breathing cycles, e.g. about 10 seconds. The leak flow rate Ql may be estimated as the product of leak conductance and a function of pressure, Pm.

### 5.4.3.1.4 Respiratory Flow Rate Estimation

In one form of the present technology, a respiratory flow rate estimation algorithm 4318 receives as an input a total flow rate, Qt, a vent flow rate, Qv, and a leak flow rate, Ql, and estimates a respiratory flow rate of air, Qr, to the patient, by subtracting the vent flow rate Qv and the estimated leak flow rate Ql from the total flow rate Qt.

### 5.4.3.1.5 Flow Rate Signal Estimation

In one form of the present technology, a flow rate signal may be estimated by a flow rate signal estimation algorithm 4317 such as to generate an estimate of a total flow rate, Qt, a vent flow rate, Qv, and a leak flow rate, Ql, and further estimates of a respiratory flow rate of air, Qr, to the patient, by subtracting the vent flow rate Qv and the estimated leak flow rate Ql from the estimated total flow rate Qt. Such a flow rate signal estimation process is described in more detail herein. Such a flow rate signal estimate may be utilized in place of a flow rate signal from a flow sensor such as if a fault is detected in the operation of a flow sensor. Similarly, such a flow rate signal estimate may be utilized to detect a fault in the operation of a flow sensor or otherwise to evaluate the accuracy of the flow rate sensor as discussed in more detail herein.

### 5.4.3.2 Therapy Engine Module

In one form of the present technology, a therapy engine module 4320 receives as inputs one or more of a pressure, Pm, in a patient interface 3000, and a respiratory flow rate of air to a patient, Qr, such as one derived from the flow rate estimate signal, and provides as an output one or more therapy parameters.

In one form of the present technology, a therapy parameter is a treatment pressure Pt.

In various forms, the therapy engine module 4320 comprises one or more of the following algorithms: phase determination 4321, waveform determination 4322, ventilation determination 4323, inspiratory flow limitation detection 4324, apnea detection 4325, inspiratory M-shape detection 4326, airway patency determination 4327, typical recent ventilation determination 4328, and therapy parameter determination 4329.

### 5.4.3.2.1 Phase Determination

In one form of the present technology, a phase determination algorithm 4321 receives as an input a signal indicative of respiratory flow rate, Qr, and provides as an output a phase Φ of a current breathing cycle of a patient 1000.

In some forms, known as discrete phase determination, the phase output Φ is a discrete variable. One implementation of discrete phase determination provides a bi-valued phase output Φ with values of either inspiration or expiration, for example represented as values of 0 and 0.5 revolutions respectively, upon detecting the start of spontaneous inspiration and expiration respectively. RPT devices 4000 that “trigger” and “cycle” effectively perform discrete phase determination, since the trigger and cycle instants are the instants at which the phase changes from expiration to inspiration and from inspiration to expiration, respectively. In one implementation of bi-valued phase determination, the phase output Φ is determined to have a discrete value of 0 (indicative of inspiration) when the respiratory flow rate Qr exceeds a “trigger threshold” (thereby triggering the RPT device 4000 to deliver a “spontaneous breath”), and a discrete value of 0.5 revolutions (indicative of expiration) when the respiratory flow rate Qr falls below a “cycle threshold” (thereby “spontaneously cycling” the RPT device 4000). In some such implementations, the trigger and cycle thresholds may vary with time during a breath according to respective trigger and cycle threshold functions. Such functions are described in the Patent Cooperation Treaty patent application number PCT/AU2005/000895, published as WO 2006/000017, to ResMed Limited, the entire contents of which are herein incorporated by reference.

In some such implementations, cycling may be prevented during a “refractory period” (denoted as Timin) after the last trigger instant, and, absent spontaneous cycling, must occur within an interval (denoted as Timax) after the last trigger instant. The values of Timin and Timax are settings of the RPT device 4000, and may be set for example by hard-coding during configuration of the RPT device 4000 or by manual entry through the input device 4220.

In other forms, known as continuous phase determination, the phase output Φ is a continuous variable, for example varying from 0 to 1 revolutions, or 0 to 2π radians. RPT devices 4000 that perform continuous phase determination may trigger and cycle when the continuous phase reaches 0 and 0.5 revolutions, respectively. In one implementation of continuous phase determination, the inspiratory time Ti and the expiratory time Te are first estimated from the respiratory flow rate Qr. The phase Φ is then determined as the half the proportion of the inspiratory time Ti that has elapsed since the previous trigger instant, or 0.5 revolutions plus half the proportion of the expiratory time Te that has elapsed since the previous cycle instant (whichever was more recent).

In some implementations, suitable for ventilation therapy (described below), the phase determination algorithm 4321 is configured to trigger even when the respiratory flow rate Qr is insignificant, such as during an apnea. As a result, the RPT device 4000 delivers “backup breaths” in the absence of spontaneous respiratory effort from the patient 1000. For such forms, known as spontaneous/timed (ST) modes, the phase determination algorithm 4321 may make use of a “backup rate” Rb. The backup rate Rb is a setting of the RPT device 4000, and may be set for example by hard-coding during configuration of the RPT device 4000 or by manual entry through the input device 4220.

A phase determination algorithm 4321 (either discrete, or continuous) may implement ST modes using the backup rate Rb in a manner known as timed backup. Timed backup may be implemented as follows: the phase determination algorithm 4321 attempts to detect the start of inspiration due to spontaneous respiratory effort, for example by comparing the respiratory flow rate Qr with a trigger threshold as described above. If the start of spontaneous inspiration is not detected within an interval after the last trigger instant whose duration is equal to the reciprocal or inverse of the backup rate Rb (an interval referred to as the backup timing threshold, Tbackup), the phase determination algorithm 4321 sets the phase output Φ to value of 0, thereby triggering the RPT device 4000 to deliver a backup breath. The phase determination algorithm 4321 then attempts to detect the start of spontaneous expiration, for example by comparing the respiratory flow rate Qr with a cycle threshold as described above. The cycle threshold for backup breaths may be different from the cycle threshold for spontaneous breaths. As with spontaneous breaths, spontaneous cycling during backup breaths may be prevented during a “refractory period” of duration Timin after the last trigger instant.

As with spontaneous breaths, if during a backup breath the start of spontaneous expiration is not detected within Timax seconds after the last trigger instant, the phase determination algorithm 4321 sets the phase output Φ to value of 0.5, thereby cycling the RPT device 4000. The phase determination algorithm 4321 then attempts to detect the start of spontaneous inspiration by comparing the respiratory flow rate Qr with a trigger threshold as described above.

### 5.4.3.2.2 Waveform Determination

In one form of the present technology, the waveform determination algorithm 4322 provides an approximately constant treatment pressure throughout a respiratory cycle of a patient.

In other forms of the present technology, the waveform determination algorithm 4322 controls the pressure generator 4140 to provide a treatment pressure Pt that varies throughout a respiratory cycle of a patient according to a waveform template.

In one form of the present technology, a waveform determination algorithm 4322 provides a waveform template Π(Φ) with values in the range [0, 1] on the domain of phase values Φ provided by the phase determination algorithm 4321 to be used by the waveform determination algorithm 4322.

In one form, suitable for either discrete or continuously-valued phase, the waveform template Π(Φ) is a square-wave template, having a value of 1 for values of phase up to and including 0.5 revolutions, and a value of 0 for values of phase above 0.5 revolutions. In one form, suitable for continuously-valued phase, the waveform template Π(Φ) comprises two smoothly curved portions, namely a smoothly curved (e.g. raised cosine) rise from 0 to 1 for values of phase up to 0.5 revolutions, and a smoothly curved (e.g. exponential) decay from 1 to 0 for values of phase above 0.5 revolutions.

In some forms of the present technology, the waveform determination algorithm 4322 selects a waveform template Π(Φ) from a library of waveform templates, dependent on a setting of the RPT device 4000. Each waveform template Π(Φ) in the library may be provided as a lookup table of values H against phase values Φ. In other forms, the waveform determination algorithm 4322 computes a waveform template Π(Φ) “on the fly” using a predetermined functional form, possibly parametrised by one or more parameters (e,g. a rise time and a fall time). The parameters of the functional form may be predetermined or dependent on a current state of the patient 1000.

In some forms of the present technology, suitable for discrete bi-valued phase of either inspiration (Φ=0 revolutions) or expiration (Φ=0.5 revolutions), the waveform determination algorithm 4322 computes a waveform template H “on the fly” as a function of both discrete phase Φ and time t measured since the most recent trigger instant. In one such form, the waveform determination algorithm 4322 computes the waveform template Π(Φ, t) in two portions (inspiratory and expiratory) as follows:

$$Π ( Φ , t ) = \{ \begin{matrix}{Π}_{i} ( t ) , & Φ = 0 \\ {Π}_{e} ( t - Ti ) , & Φ = 0. 5\end{matrix} \begin{matrix} \\ \end{matrix}$$

where Πᵢ(t) and Πₑ(t) are inspiratory and expiratory portions of the waveform template Π(Φ, t).

In one such form, the inspiratory portion IVO of the waveform template is a smooth rise from 0 to 1 in two continuous sections:
a linear rise up to ⅔ for the first half of a parameter known as the “time scale”;
a parabolic rise up to 1 for the second half of the time scale.

The “rise time” of such an inspiratory portion Πᵢ(t) may be defined as the time taken for Πᵢ(t) to rise to a value of 0.875.

The expiratory portion He(t) of the waveform template is a smooth fall from 1 to 0 in two continuous parabolic sections, with an inflection point between 25% and 50% of the time scale. The “fall time” of such an expiratory portion Πₑ(t) may be defined as the time taken for Πₑ(t) to fall to a value of 0.125.

### 5.4.3.2.3 Ventilation Determination

In one form of the present technology, a ventilation determination algorithm 4323 receives an input a respiratory flow rate Qr, which may be derived from an estimated flow rate signal as previously described, and determines a measure Vent indicative of current patient ventilation.

In some implementations, the ventilation determination algorithm 4323 computes Vent as an “instantaneous ventilation” Vint, which is half the absolute value of the respiratory flow rate signal Qr.

In some implementations, the ventilation determination algorithm 4323 computes Vent as a “very fast ventilation” VveryFast by filtering the instantaneous ventilation Vint by a low-pass filter such as a fourth order Bessel low-pass filter with a corner frequency of approximately 0.10 Hz. This is equivalent to a time constant of approximately ten seconds.

In some implementations, the ventilation determination algorithm 4323 computes a Vent as a “fast ventilation” Vfast by filtering the instantaneous ventilation Vint by a low-pass filter such as a fourth order Bessel low-pass filter with a corner frequency of approximately 0.05 Hz. This is equivalent to a time constant of approximately twenty seconds.

In some implementations of the present technology, the ventilation determination algorithm 4323 determines Vent as a measure of alveolar ventilation. Alveolar ventilation is a measure of how much air is actually reaching the gas exchange surfaces of the respiratory system in a given time. Because the respiratory system of the patient includes a significant “anatomical dead space”, i.e. volume in which gas exchange does not take place, the alveolar ventilation is less than the “gross” ventilation values that the above calculations that operate directly on the respiratory flow rate Qr will produce, but is a more accurate measure of the respiratory performance of a patient.

In such implementations, the ventilation determination algorithm 4323 may determine the instantaneous alveolar ventilation to be either zero or half the absolute value of the respiratory flow rate Qr. The conditions under which the instantaneous alveolar ventilation is zero are:
When the respiratory flow rate changes from non-negative to negative, or
When the respiratory flow rate changes from negative to non-negative, and
After the respiratory flow rate has changed sign, for the period during which the absolute value of the integral of the respiratory flow rate Qr is less than the patient's anatomical dead space volume.

The patient's anatomical dead space volume may be a setting of the RPT device 4000, set for example by hard-coding during configuration of the RPT device 4000 or by manual entry through the input device 4220.

In some such implementations, the ventilation determination algorithm 4323 may compute Vent as a “very fast alveolar ventilation” and/or a “fast alveolar ventilation” by low-pass filtering the instantaneous alveolar ventilation using the respective low-pass filters described above.

In what follows the word “alveolar” is omitted but it may be assumed to be present in some implementations of the therapy engine module 4320. That is, mentions of “ventilation” and “tidal volume” in the subsequent description may be taken to apply to alveolar ventilation and alveolar tidal volume as well as “gross” ventilation and tidal volume.

### 5.4.3.2.4 Inspiratory Flow Limitation Determination

In one form of the present technology, the therapy engine module 4320 executes one or more algorithms to determine the extent of flow limitation, sometimes referred to as partial upper airway obstruction, in the inspiratory portion of the respiratory flow rate waveform (herein sometimes shortened to the “inspiratory waveform”). In one form, the flow limitation determination algorithm 4324 receives as an input a respiratory flow rate signal Qr, which may be derived from an estimated flow rate signal as previously described, and provides as an output a measure of the extent to which each inspiratory waveform exhibits flow limitation.

A normal inspiratory waveform is rounded, close to sinusoidal in shape (see FIG. 6A). With sufficient upper airway muscle tone (or EPAP), the airway acts essentially as a rigid tube, where flow increases in response to increased breathing effort (or external ventilatory assistance). In some situations (e.g. sleep, sedation) the upper airway may be collapsible, such as in response to sub-atmospheric pressure within it from breathing effort, or even from applied ventilation. This can lead to either full obstruction (apneas), or a phenomenon known as ‘flow limitation’. The term ‘flow limitation’ includes behaviour where increased breathing effort simply induces increased narrowing of the airway, such that inspiratory flow becomes limited at a constant value, independent of effort (“Starling resistor behaviour”). Therefore, the inspiratory flow rate curve exhibits a flattened shape (see FIG. 6B).

In reality, upper airway behaviour is even more complicated, and there is a wide variety of flow shapes that are indicative of upper airway-related inspiratory flow limitation, and an even wider variety in the presence of external ventilatory assistance (see FIGS. 6C to 6F). For this reason, the flow limitation determination algorithm 4324 may respond to one or more of the following kinds of inspiratory flow limitation: “classical flatness” (see FIG. 6B), “chairness” (see FIG. 6C), and “reverse chairness” (see FIG. 6D). (“M-shape” (see FIGS. 6E and 6F) is dealt with separately, using M-shape detection algorithm 4326.)

### 5.4.3.2.5 M-Shape Detection

In one form of the present technology, the therapy engine 4320 module executes one or more algorithms to detect “M-shape” in the inspiratory waveform. In one form, the M-shape detection algorithm 4326 receives as an input a respiratory flow rate signal Qr and provides as an output a measure indicative of the extent to which each inspiratory waveform exhibits M-shape.

M-shaped inspiratory waveforms with tidal volumes or other breathwise ventilation values not much greater than typical recent values are indicative of flow limitation. Such inspiratory waveforms have a relatively rapid rise and fall and a dip or “notch” in flow approximately in the centre, the dip being due to flow limitation (see FIGS. 6E and 6F). At higher tidal volumes or breathwise ventilation values, such waveforms are generally behavioural, i.e. micro-arousals during sleep, or sighs, and are not indicative of flow limitation.

To detect M-shaped waveforms, the M-shape detection algorithm 4326 determines the similarity of the inspiratory waveform to a waveform which is broadly M-shaped.

### 5.4.3.2.6 Apnea Detection

In one form of the present technology, the therapy engine module 4320 executes an apnea detection algorithm 4325 to detect apneas.

In one form, the apnea detection algorithm 4325 receives as an input a respiratory flow rate signal Qr and provides as an output a series of events indicating starts and ends of detected apneas.

### 5.4.3.2.7 Typical Recent Ventilation Determination

In one form of the present technology, the central controller 4230 takes as input the measure of current ventilation, Vent, and executes one or more typical recent ventilation determination algorithms 4328 for the determination of a value Vtyp indicative of the typical recent ventilation of the patient 1000.

The typical recent ventilation Vtyp is the value around which the distribution of the measure of current ventilation Vent over multiple time instants over some predetermined timescale tends to cluster, that is, a measure of the central tendency of the measure of current ventilation over recent history. In one implementation of the typical recent ventilation determination algorithm 4328, the recent history is of the order of several minutes, but in any case should be longer than the timescale of Cheyne-Stokes waxing and waning cycles. The typical recent ventilation determination algorithm 4328 may use any of the variety of well-known measures of central tendency to determine the typical recent ventilation Vtyp from the measure of current ventilation, Vent. One such measure is the output of a low-pass filter on the measure of current ventilation Vent, with time constant equal to one hundred seconds.

### 5.4.3.2.8 Airway Patency Determination

In one form of the present technology, the central controller 4230 executes an airway patency determination algorithm 4327 for the determination of airway patency. In some implementations, the airway patency determination algorithm 4327 returns either “closed” or “open”, or equivalent Boolean values, e.g. “true” indicating closed and “false” indicating open.

### 5.4.3.2.9 Determination of Therapy Parameters

In some forms of the present technology, the central controller 4230 executes one or more therapy parameter determination algorithms 4329 for the determination of one or more therapy parameters using the values returned by one or more of the other algorithms in the therapy engine module 4320.

In one form of the present technology, the therapy parameter is an instantaneous treatment pressure Pt. In one implementation of this form, the therapy parameter determination algorithm 4329 determines the treatment pressure Pt as follows:

Pt=AΠ(Φ,t)+P₀ (1)

where A is the amount of “pressure support”, Π(Φ, t) is the waveform template value (in the range 0 to 1) at the current values Φ of phase and t of time, and P₀ is a base pressure.

By determining the treatment pressure Pt using equation (1) and applying it as a set point in the controller 4230 of the RPT device 4000, the therapy parameter determination algorithm 4329 oscillates the treatment pressure Pt in synchrony with the spontaneous respiratory effort of the patient 1000. That is, based on the typical waveform templates Π(Φ) described above, the therapy parameter determination algorithm 4329 increases the treatment pressure Pt at the start of, or during, or inspiration and decreases the treatment pressure Pt at the start of, or during, expiration. The (non-negative) pressure support A is the amplitude of the oscillation.

If the waveform determination algorithm 4322 provides the waveform template Π(Φ) as a lookup table, the therapy parameter determination algorithm 4329 applies equation (1) by locating the nearest lookup table entry to the current value Φ of phase returned by the phase determination algorithm 4321, or by interpolation between the two entries straddling the current value Φ of phase.

The values of the pressure support A and the base pressure P₀ may be determined by the therapy parameter determination algorithm 4329 depending on the chosen respiratory pressure therapy mode in the manner described below.

### 5.4.3.3 Therapy Control Module

Therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of air whose mask pressure Pm at the patient interface 3000 is equal to the treatment pressure Pt or whose interface flow rate Ft at the interface is equal to a treatment flow TFₜ

### 5.4.3.4 Detection of Fault Conditions

In one form of the present technology, the central controller 4230 executes one or more methods for the detection of fault conditions. The fault conditions detected by the one or more methods may include at least one of the following:
Power failure (no power, or insufficient power)
Transducer fault detection
Failure to detect the presence of a component
Operating parameters outside recommended ranges (e.g. pressure, flow rate, temperature, PaO₂)
Failure of a test alarm to generate a detectable alarm signal.
Significant inequality of flow rate signal from flow sensor and flow rate estimate signal from flow rate signal estimation 4137 process

Upon detection of the fault condition, the corresponding algorithm signals the presence of the fault by one or more of the following:
Initiation of an audible, visual &/or kinetic (e.g. vibrating) alarm
Sending a message to an external device
Logging of the incident
change of control parameters as described in more detail herein

### 5.5 Humidifier

In one form of the present technology there is provided a humidifier 5000 (e.g., as shown in FIGS. 4B) to change the absolute humidity of air or gas for delivery to a patient relative to ambient air. Typically, the humidifier 5000 is used to increase the absolute humidity and increase the temperature of the flow of air (relative to ambient air) before delivery to the patient's airways. In some versions, the humidifier may be incorporated into the housing of the RPT device 4000.

The humidifier 5000 may comprise a tub or humidifier reservoir, a humidifier inlet to receive a flow of air, and a humidifier outlet to deliver a humidified flow of air. In some forms, an inlet and an outlet of the humidifier reservoir of the humidifier may be removably applied to a humidifier inlet and the humidifier outlet respectively such as when the reservoir is configured as a removable tub. The humidifier 5000 may further comprise a humidifier base with a chassis of the external housing of the humidifier, which may be adapted to receive the humidifier reservoir and comprise a heating element (not shown) for heating the reservoir contents. As illustrated in FIG. 4B, the humidifier outlet may be coupled with a coupler or adaptor 4171A for pneumatic coupling with a tube of a patient circuit 4170.

### 5.6 Respiratory Pressure Therapy Modes

Various respiratory pressure therapy modes may be implemented by the RPT device 4000 depending on the values of the parameters A and P₀ in the treatment pressure equation (1) used by the therapy parameter determination algorithm 4329 in one form of the present technology.

### 5.6.1 CPAP Therapy

In some implementations, the pressure support A is identically zero, so the treatment pressure Pt is identically equal to the base pressure P₀ throughout the respiratory cycle. Such implementations are generally grouped under the heading of CPAP therapy. In such implementations, there is no need for the therapy engine module 4320 to determine phase Φ or the waveform template Π(Φ).

### 5.6.2 Ventilation Therapy

In other implementations, the value of pressure support A in equation (1) may be positive. Such implementations are known as ventilation therapy. In some forms of ventilation therapy, known as fixed pressure support ventilation therapy, the pressure support A is fixed at a predetermined value, e.g. 10 cmH₂O. The predetermined value of pressure support A is a setting of the RPT device 4000, and may be set for example by hard-coding during configuration of the RPT device 4000 or by manual entry through the input device 4220. In some versions, the pressure may be bi-level such as where a higher pressure is delivered during patient inspiration and a lower pressure is delivered during patient expiration

The value of the pressure support A may be limited to a range defined as [Amin, Amax]. The pressure support limits Amin and Amax are settings of the RPT device 4000, set for example by hard-coding during configuration of the RPT device 4000 or by manual entry through the input device 4220. A minimum pressure support Amin of 3 cmH₂O is of the order of 50% of the pressure support required to perform all the work of breathing of a typical patient in the steady state. A maximum pressure support Amax of 12 cmH₂O is approximately double the pressure support required to perform all the work of breathing of a typical patient, and therefore sufficient to support the patient's breathing if they cease making any efforts, but less than a value that would be uncomfortable or dangerous.

### 5.7 Glossary

For the purposes of the present technology disclosure, in certain forms of the present technology, one or more of the following definitions may apply. In other forms of the present technology, alternative definitions may apply.

### 5.7.1 General

Air: In certain forms of the present technology, air may be taken to mean atmospheric air, and in other forms of the present technology air may be taken to mean some other combination of breathable gases, e.g. atmospheric air enriched with oxygen.
Ambient: In certain forms of the present technology, the term ambient will be taken to mean (i) external of the treatment system or patient, and (ii) immediately surrounding the treatment system or patient or outside of the pneumatic path of the RPT device.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.
Respiratory Pressure Therapy (RPT): The application of a supply of air to an entrance to the airways at a treatment pressure that is typically positive with respect to atmosphere.
Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during expiration, and slightly lower during inspiration. In some forms, the pressure will vary between different respiratory cycles of the patient, for example, being increased in response to detection of indications of partial upper airway obstruction, and decreased in the absence of indications of partial upper airway obstruction.
Patient: A person, whether or not they are suffering from a respiratory disorder.
Automatic Positive Airway Pressure (APAP) therapy: CPAP therapy in which the treatment pressure is automatically adjustable, e.g. from breath to breath, between minimum and maximum limits, depending on the presence or absence of indications of SDB events.

### 5.7.2 Aspects of the Respiratory Cycle

Apnea: According to some definitions, an apnea is said to have occurred when flow falls below a predetermined threshold for a duration, e.g. 10 seconds. A closed apnea will be said to have occurred when, despite patient effort, some obstruction of the airway does not allow air to flow. An open apnea will be said to have occurred when an apnea is detected that is due to a reduction in breathing effort, or the absence of breathing effort, despite the airway being open (patent). A mixed apnea occurs when a reduction or absence of breathing effort coincides with an obstructed airway.
Breathing rate: The rate of spontaneous respiration of a patient, usually measured in breaths per minute.
Duty cycle, or inspiratory fraction: The ratio of inspiratory time, Ti, to total breath time, Ttot.
Effort (breathing): Breathing effort will be said to be the work done by a spontaneously breathing person attempting to breathe.
Expiratory portion of a breathing cycle: The period from the start of expiratory flow to the start of inspiratory flow.
Flow limitation: Flow limitation will be taken to be the state of affairs in a patient's respiration where an increase in effort by the patient does not give rise to a corresponding increase in flow. Where flow limitation occurs during an inspiratory portion of a breathing cycle it may be described as inspiratory flow limitation. Where flow limitation occurs during an expiratory portion of the breathing cycle it may be described as expiratory flow limitation.

Types of flow-limited inspiratory waveforms:
(i) (Classically) Flattened: Having a rise followed by a relatively flat portion, followed by a fall.
(ii) M-shaped: Having two local peaks, one at the early part, and one at the late section, and a relatively flat portion between the two peaks.
(iii) Chair-shaped: Having a single local peak, the peak being at the early part, followed by a relatively flat portion.
(iv) Reverse-chair shaped: Having a relatively flat portion followed by single local peak, the peak being at the late section.
Hypopnea: A reduction in flow, but not a cessation of flow. In one form, a hypopnea may be said to have occurred when there is a reduction in flow below a threshold rate for a duration. A central hypopnea may be said to have occurred when a hypopnea is detected that is due to a reduction in breathing effort.
Hyperpnea: An increase in flow to a level higher than normal flow rate.
Hypoventilation: Hypoventilation is said to occur when the amount of gas exchange taking place over some timescale is below the current requirements of the patient.
Hyperventilation: Hyperventilation is said to occur when the amount of gas exchange taking place over some timescale is above the current requirements of the patient.
Inspiratory portion of a breathing cycle: The period from the start of inspiratory flow to the start of expiratory flow will be taken to be the inspiratory portion of a breathing cycle.
Patency (airway): The degree of the airway being open, or the extent to which the airway is open. A patent airway is open. Airway patency may be quantified, for example with a value of one (1) being patent, and a value of zero (0), being closed (obstructed).
Positive End-Expiratory Pressure (PEEP): The pressure above atmosphere in the lungs that exists at the end of expiration.
Peak flow rate (Qpeak): The maximum value of flow rate during the inspiratory portion of the respiratory flow rate waveform.
Respiratory flow rate, airflow rate, patient airflow rate, respiratory airflow rate (Qr): These synonymous terms may be understood to refer to the RPT device's estimate of respiratory airflow rate, as opposed to “true respiratory flow” or “true respiratory airflow”, which is the actual respiratory flow rate experienced by the patient, usually expressed in litres per minute.
Tidal volume (Vt): The volume of air inspired or expired per breath during normal breathing, when extra effort is not applied. This quantity may be more specifically defined as inspiratory tidal volume (Vi) or expiratory tidal volume (Ve).
Inspiratory Time (Ti): The duration of the inspiratory portion of the respiratory flow rate waveform.
Expiratory Time (Te): The duration of the expiratory portion of the respiratory flow rate waveform.
Total (breath) Time (Ttot): The total duration between the start of the inspiratory portion of one respiratory flow rate waveform and the start of the inspiratory portion of the following respiratory flow rate waveform.
Typical recent ventilation: The value of ventilation around which recent values over some predetermined timescale tend to cluster, that is, a measure of the central tendency of the recent values of ventilation.
Upper airway obstruction (UAO): includes both partial and total upper airway obstruction. This may be associated with a state of flow limitation, in which the level of flow increases only slightly or may even decrease as the pressure difference across the upper airway increases (Starling resistor behaviour).
Ventilation (Vent): A measure of the total amount of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 5.7.3 RPT Device Parameters

Flow rate: The instantaneous volume (or mass) of air delivered per unit time. While flow rate and ventilation have the same dimensions of volume or mass per unit time, flow rate is measured over a much shorter period of time. In some cases, a reference to flow rate will be a reference to a scalar quantity, namely a quantity having magnitude only. In other cases, a reference to flow rate will be a reference to a vector quantity, namely a quantity having both magnitude and direction. Where it is referred to as a signed quantity, a flow rate may be nominally positive for the inspiratory portion of a breathing cycle of a patient, and hence negative for the expiratory portion of the breathing cycle of a patient. Flow rate will be given the symbol Q. ‘Flow rate’ is sometimes shortened to simply ‘flow’. Total flow rate, Qt, is the flow rate of air leaving the RPT device. Vent flow rate, Qv, is the flow rate of air leaving a vent to allow washout of expired gases. Leak flow rate, Ql, is the flow rate of leak from a patient interface system. Respiratory flow rate, Qr, is the flow rate of air that is received into the patient's respiratory system.
Leak: The word leak will be taken to be an unintended flow of air. In one example, leak may occur as the result of an incomplete seal between a mask and a patient's face. In another example leak may occur in a swivel elbow to the ambient.
Pressure: Force per unit area. Pressure may be measured in a range of units, including cmH₂O (centimetres of water), g-f/cm², and hectopascals (hPa). 1 cmH₂O is equal to 1 g-f/cm² and is approximately 0.98 hPa. In this specification, unless otherwise stated, pressure is given in units of cmH₂O. The pressure in the patient interface is given the symbol Pm, while the treatment pressure, which represents a target value to be achieved by the mask pressure Pm at the current instant of time, is given the symbol Pt.

### 5.7.4 Terms for Ventilators

Backup rate: A parameter of a ventilator that establishes the minimum breathing rate (typically in number of breaths per minute) that the ventilator will deliver to the patient, if not triggered by spontaneous respiratory effort.
Cycling: The termination of a ventilator's inspiratory phase. When a ventilator delivers a breath to a spontaneously breathing patient, at the end of the inspiratory portion of the breathing cycle, the ventilator is said to be cycled to stop delivering the breath.
Expiratory positive airway pressure (EPAP): a base pressure, to which a pressure varying within the breath is added to produce the desired mask pressure which the ventilator will attempt to achieve at a given time.
End expiratory pressure (EEP): Desired mask pressure which the ventilator will attempt to achieve at the end of the expiratory portion of the breath. If the pressure waveform template Π(Φ) is zero-valued at the end of expiration, i.e. Π(Φ)=0 when Φ=1, the EEP is equal to the EPAP.
Inspiratory positive airway pressure (IPAP): Maximum desired mask pressure which the ventilator will attempt to achieve during the inspiratory portion of the breath.
Pressure support: A number that is indicative of the increase in pressure during ventilator inspiration over that during ventilator expiration, and generally means the difference in pressure between the maximum value during inspiration and the base pressure (e.g., PS=IPAP− EPAP). In some contexts pressure support means the difference which the ventilator aims to achieve, rather than what it actually achieves.
Servo-ventilator: A ventilator that measures patient ventilation, has a target ventilation, and which adjusts the level of pressure support to bring the patient ventilation towards the target ventilation.
Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.
Swing: Equivalent term to pressure support.
Triggering: When a ventilator delivers a breath of air to a spontaneously breathing patient, it is said to be triggered to do so at the initiation of the respiratory portion of the breathing cycle by the patient's efforts.
Ventilator: A mechanical device that provides pressure support to a patient to perform some or all of the work of breathing.

### 5.7.5 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.
Larynx: The larynx, or voice box houses the vocal folds and connects the inferior part of the pharynx (hypopharynx) with the trachea.
Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.
Nasal cavity: The nasal cavity (or nasal fossa) is a large air-filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.
Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 5.8 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” (etc.) may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 5.9 REFERENCE LABEL LIST

patient 1000
patient interface 3000
seal-forming structure 3100
plenum chamber 3200
structure 3300
vent 3400
connection port 3600
forehead support 3700
RPT device 4000
aperture 4001
external housing 4010
upper portion 4012
portion 4014
panel 4015
chassis 4016
frame 4016
handle 4018
pneumatic block 4020
pneumatic components 4100
air filter 4110
inlet air filter 4112
outlet air filter 4114
inlet muffler 4122
outlet muffler 4124
flow rate signal estimation 4137
pressure generator 4140
blower 4142
motor 4144
patient circuit 4170
supplemental oxygen 4180
electrical components 4200
main PCBA 4202
sensor PCBA 4203
power supply 4210
input devices 4220
controller 4230
clock 4232
therapy device controller 4240
protection circuits 4250
memory 4260
transducers 4270
pressure sensor 4272
sound sensor 4273
flow rate sensor 4274
gas temperature sensor 4275
motor speed transducer 4276
data communication interface 4280
remote external communication network 4282
local external communication network 4284
remote external device 4286
local external device 4288
output device 4290
display driver 4292
display 4294
algorithms 4300
pre-processing module 4310
pressure compensation algorithm 4312
vent flow rate estimation algorithm 4314
leak flow rate estimation algorithm 4316
flow rate signal estimation algorithm 4317
respiratory flow rate estimation algorithm 4318
therapy engine module 4320
phase determination algorithm 4321
waveform determination algorithm 4322
ventilation determination algorithm 4323
flow limitation determination algorithm 4324
inspiratory flow limitation detection 4324
apnea detection algorithm 4325
M-shape detection algorithm 4326
airway patency determination algorithm 4327
typical recent ventilation determination algorithm 4328
therapy parameter determination algorithm 4329
therapy control module 4330
housing 4331
pneumatic block 4333
inlet 4335
outlet 4337
pneumatic block 4340
sensor module assembly 4400
sensor manifold assembly 4400
sensor module 4402
wired connector 4403
flow manifold 4404
main seal 4406
sensor seal 4408
block coupling seal 4410
coupling ridges 4411
first coupling end 4412
second coupling end 4414
coupling end seal(s) 4415
housing panel 4416
alignment tab 4417
gas port 4418
tab recess 4419
support plate 4422
fixing post 4424
peak 4426
apertures 4427
alignment post 4428
pin 4430
aperture 4432
peak surface 4434
rim 4436
annular cavity 4437
seal surface 4438
outlet 4440
annular nipple 4442
flow sensor seal 4444
pressure sensor seal 4448
seal spacing extension 4450
UI assembly 4502
top case 4504
end cover 4508
bottom case 4510
battery door 4512
inlet tube 4514
main frame 4516
frame cup 4518
connector module assembly 4520
connector infill 4522
branding plaque 4524
humidifier 5000
humidifier inlet 5002
aperture 5003
humidifier outlet 5004
humidifier end cap 5005
humidifier top case 5007
humidifier bottom case 5009
latch 5013
latch button 5015
chassis 5017
adaptor 5019
tube 5021
humidifier reservoir 5110
humidifier controller 5250

1. A sensor module assembly for a respiratory therapy apparatus that includes a pneumatic block assembly, the sensor module assembly comprising:
a flow manifold, the flow manifold comprising an inlet and an outlet and a main pneumatic path between the inlet and the outlet; and
a sensor module comprising a plurality of sensors on a printed circuit board, wherein the sensor module is pneumatically and fixedly coupled to the flow manifold.

2. The sensor module assembly of claim 1 wherein the sensor module assembly is configured to be substantially secured for use within the respiratory therapy apparatus by its pneumatic couplings.

3. The sensor module assembly of any one of claims 1 to 2 wherein the inlet of the flow manifold is configured for removable coupling with an outlet associated with the pneumatic block assembly and wherein the outlet of the flow manifold is configured for removable coupling with a pneumatic path associated with a housing structure of the respiratory therapy apparatus, the pneumatic path being adapted for supplying breathable gas to a patient interface.

4. The sensor module assembly of claim 3 wherein the inlet of the flow manifold is configured for removable coupling with an outlet of the pneumatic block assembly and wherein the outlet of the flow manifold is configured for removable coupling with a pneumatic path of a housing cover panel of the respiratory therapy apparatus, the pneumatic path being adapted for supplying breathable gas to a patient interface.

5. The sensor module assembly of any one of claims 1 to 4 wherein the flow manifold comprises a cylinder, the cylinder comprising the main pneumatic path, wherein the cylinder has a central axis from the inlet of the flow manifold to the outlet of the flow manifold, the central axis being along a length of the cylinder.

6. The sensor module assembly of any one of claims 1 to 5 wherein the flow manifold further comprises a plurality of gas ports, the plurality of gas ports being pneumatically coupled to the main pneumatic path.

7. The sensor module assembly of claim 6 wherein each of the plurality of gas ports are cylindrical, and wherein each cylindrical gas port has a central axis.

8. The sensor module assembly of claim 7 wherein the central axes of the cylindrical gas ports are substantially parallel.

9. The sensor module assembly of claim 7 wherein the central axes of the cylindrical gas ports are generally perpendicular to the central axis of the cylinder of the flow manifold.

10. The sensor module assembly of any one of claims 6 to 9 wherein the flow manifold further comprises a support plate, the support plate configured as a shelf at a side of the flow manifold and integrated with a set of the gas ports.

11. The sensor module assembly of claim 10 wherein the set of the gas ports comprises first and second ports configured for sensing a flow rate within the flow manifold.

12. The sensor module assembly of any one of claims 1 to 10 wherein the flow manifold further comprises a plurality of mounting posts.

13. The sensor module assembly of claim 12 wherein a plurality of the mounting posts are configured on a first side of the main pneumatic path of the flow manifold and a plurality of gas ports are configured on a second side the main pneumatic path, the second side being an opposing side of the main pneumatic path relative the first side.

14. The sensor module assembly of claim 13 wherein the plurality of mounting posts and the plurality of gas ports are configured to support the sensor module.

15. The sensor module assembly of claim 14 wherein the plurality of mounting posts are configured to support the printed circuit board and the plurality of gas ports are configured to support a sensor of the plurality of sensors.

16. The sensor module assembly of any one of claims 12 to 15, wherein the plurality of mounting posts comprises at least one fixing post configured for receiving a fastener of the sensor module for fixedly coupling the sensor module to the flow manifold.

17. The sensor module assembly of any one of claims 12 to 16, wherein each of the mounting posts has a central axis, wherein the central axes of the mounting posts are substantially parallel.

18. The sensor module assembly of claim 17, when dependent on claim 6, wherein the central axes of the mounting posts are generally parallel to the central axes of the plurality of gas ports.

19. The sensor module assembly of any one of claims 1 to 18 wherein the inlet of the flow manifold comprises a female coupling and the outlet of the pneumatic block assembly comprises a male coupling.

20. The sensor module assembly of any one of claims 1 to 18 wherein the inlet of the flow manifold comprises a male coupling and the outlet of the pneumatic block assembly comprises a female coupling.

21. The sensor module assembly of any one of claims 19 to 20 further comprising a tubular seal, the tubular seal configured for mating between the male coupling and the female coupling, the tubular seal providing a pneumatic seal between the inlet of the flow manifold and the outlet of the pneumatic block assembly.

22. The sensor module assembly of any one of claims 19 to 20 further comprising a face seal, the face seal configured for mating between the male coupling and the female coupling, the face seal providing a pneumatic seal between the inlet of the flow manifold and the outlet of the pneumatic block assembly.

23. The sensor module assembly of claim 21 wherein the tubular seal is configured for (1) insertion within the female coupling and (2) receiving the male coupling within the tubular seal.

24. The sensor module assembly of any one of claims 21 to 23, when dependent on claim 6, wherein the tubular seal comprises a multiport seal unit configured to pneumatically seal the plurality of gas ports of the flow manifold with the plurality of sensors of the sensor module.

25. The sensor module assembly of claim 24 wherein the tubular seal and multiport seal unit are overmoulded to the flow manifold.

26. The sensor module assembly of any one of claims 1 to 23 further comprising a main seal, the main seal configured for sealing coupling of the outlet of the flow manifold with the pneumatic path of the housing cover panel.

27. The sensor module assembly of any one of claims 6 to 23 and 25 further comprising a multiport seal unit, the multiport seal unit configured to (1) pneumatically seal the plurality of gas ports of the flow manifold with the plurality of sensors of the sensor module, and (2) pneumatically seal the outlet of the flow manifold with the pneumatic path of a housing cover of the respiratory therapy apparatus.

28. The sensor module assembly of claim 27 wherein the multiport seal comprises a flow sensor seal and a pressure sensor seal separated by a first seal spacing extension.

29. The sensor module assembly of claim 28 wherein the multiport seal comprises a main seal separated from the flow sensor seal and the pressure sensor seal by a second seal spacing extension, the main seal configured for sealing coupling of the outlet of the flow manifold with the pneumatic path of the housing cover panel.

30. The sensor module assembly of any one of claims 1 to 29 wherein the flow manifold is a moulded component formed of a thermoplastic material.

31. The sensor module assembly of any one of claims 1 to 30 wherein the plurality of sensors includes a pressure sensor and a flow sensor.

32. The sensor module assembly of claim 31 wherein the plurality of sensors further includes an additional pressure sensor, a microphone or a temperature sensor.

33. The sensor module assembly of any one of claims 1 to 32 wherein the pneumatic block assembly comprises a housing that includes a volute with a motor and impeller configured to produce air at a positive pressure at the outlet of the pneumatic block.

34. The respiratory therapy apparatus for providing a breathable gas to a patient interface for a respiratory therapy, the respiratory therapy apparatus comprising:
a sensor module assembly of any one of claims 1 to 33;
the pneumatic block assembly; and
an external housing to receive the sensor module assembly and the pneumatic block assembly.

35. The respiratory therapy apparatus of claim 34, when dependent on claim 3, wherein the external housing comprises the housing cover panel.

36. The respiratory therapy apparatus of claim 34 wherein the sensor module assembly is configured for seal-based securement within the external housing.
